(19) United States
(12) Patent Application Publication
(10) Pub. No.: US 2005/0014264 A1
(43) Pub. Date: Jan. 20, 2005

(54) Method of introducing siRNA into adipocytes

(75) Inventors: Michael P. Czech, Westborough, MA (US); Qiong L. Zhou, Shrewsbury, MA (US); Zhen Y. Jiang, Shrewsbury, MA (US)

Correspondence Address: LAHIVE & COCKFIELD, LLP., 28 STATE STREET, BOSTON, MA, 02109, US

(73) Assignee: UNIVERSITY OF MASSACHUSETTS, Worcester, MA (US)

(21) Appl. No.: 10/735,461

(22) Filed: Dec. 11, 2003

Related U.S. Application Data
(60) Provisional application No. 60/432,427, filed on Dec. 11, 2002.

(51) Int. Cl. C12N 15/87

(52) U.S. Cl. 435/459

(57) ABSTRACT

In general, it has been difficult to introduce nucleic acid sequences into adipocytes. Methods are described for introducing nucleic acids such as siRNAs into adipocytes, including the introduction of sequences targeting Akt1, Akt2 and Myo1c. Methods for identifying genes involved in glucose transport and for identifying insulin response modulators are described. Therapeutic methods are also provided.

### RELATED APPLICATIONS

This application claims the benefit of U.S. Provisional patent application Ser. No. 60/432,427, entitled “Method of Introducing siRNA into Adipocytes”, filed Dec. 11, 2002. The entire content of the above-reference patent application is hereby incorporated by this reference.

### GOVERNMENT RIGHTS

This invention was made at least in part with government support under Grant No. DK30648 awarded by the NIH. The government may have rights in this invention.

### BACKGROUND

Cultured adipocytes represent a key model system for studying insulin action on glucose transport. No other cell type in culture expresses such high concentrations of the glucose transporter GLUT4 and responds as robustly to insulin by increasing glucose transport. This action of insulin on GLUT4 is central to type 2 diabetes because insulin resistance in fat and muscle are primary defects that lead to this disease. Discovery of components of the insulin signaling pathway are crucial to understanding insulin resistance and to providing potential drug targets for therapies for type 2 diabetes.

Discovery of such insulin signaling components is enhanced by ablating the expression of specific genes encoding proteins suspected of being involved in this pathway. No method has been reliably able to achieve specific ablation of gene or protein expression in adipocytes. These cells are difficult to work with and are not easily transfected with reagents that work in other cells such as fibroblasts.

Glucose homeostasis is controlled in part through regulation of glucose transport into muscle and fat cells by insulin. Akt protein kinases (also known as protein kinase B (PKB)) downstream of PI 3-kinase have been implicated in this insulin signaling pathway, but results with Akt1^{-/-} and Akt2^{-/-} mice have been equivocal. Akt is among the downstream effectors strongly implicated in linking insulin signaling through a PI 3-kinase-dependent pathway to components that regulate GLUT4 trafficking. This role for Akt is based upon studies with cultured cells and a gene knockout mouse model (Kohn et al., 1996, J. Biol. Chem. 49:31372; Hill et al., 1999, Mol. Cell. Biol. 19:7771; Wang et al., 1999, Mol. Cell. Biol. 19:4008; Ueki et al., 1998, J. Biol. Chem. 273:5315; Cho et al., 2001 (a), Science 292:1728). However, this important issue has been controversial (Kotani et al., 1998, Mol. Cell. Biol. 18:6971) and recent data from mice lacking the Akt1 isoform showed no compromise in insulin sensitivity (Cho et al., 2001 (b), J. Biol. Chem. 276:38349). Skeletal muscle from mice deficient in Akt2, the predominant isoform expressed in muscle and fat, is only modestly less sensitive to low levels of insulin and actually responds normally to maximal doses of the hormone (Cho et al., 2001 (a), supra). The diabetes observed in these mice may be largely due to the dramatic attenuation of insulin action on liver gluconeogenesis (Cho, 2001 (a), supra).

### SUMMARY

The invention relates, in part, to the discovery of a method for introducing siRNA into adipocytes. The method is demonstrated using siRNAs that target several different sequences, including Akt1, Akt2, CISK and Myo1c.

Accordingly, the invention includes a method of introducing a nucleic acid into an adipocyte. The method includes the steps of obtaining an adipocyte; contacting the adipocyte with a nucleic acid molecule, to form a mixture; and electroporating the mixture. The nucleic acid can be an siRNA, for example, an siRNA targeted to an Akt1, Akt2, CISK or Myo1c nucleic acid sequence. In general, the electroporation is carried out at about 0.18 kV and about 960 μF capacitance and at room temperature. In some embodiments, the invention includes a method of identifying a gene that affects glucose transport. The method includes the steps of providing an adipocyte; introducing into the adipocyte an siRNA targeted against the gene using electroporation as described herein; culturing the cell under conditions suitable for expression of the targeted gene; and assaying glucose transport in the cell, such that a reduction in glucose transport indicates that the targeted gene affects glucose transport.

In another embodiment, the invention includes a method of inhibiting Akt1 or Akt2 expression or activity in a cell. The method includes the steps of obtaining a cell that expresses Akt1 or Akt2 and introducing into the cell an siRNA targeted to an Akt1 nucleic acid sequence or targeted to an Akt2 nucleic acid sequence (for example, as depicted in FIG. 3A). In some embodiments the cell is an adipocyte. In some aspects, both Akt1 and Akt2 expression or activity are inhibited using the method. In this aspect, a siRNA targeted to Akt1 and a siRNA targeted to Akt2 can be introduced into the cell. Alternatively, siRNA that targets both Akt1 and Akt2 can be introduced into the cell.

The invention also includes a method of inhibiting insulin-mediated GSK3α phosphorylation. The method includes the steps of obtaining an insulin-sensitive cell, and contacting the cell with an agent that inhibits Akt2 expression or activity. In some aspects, the agent is an siRNA targeted to Akt2.

In another embodiment, the invention relates to a method of inhibiting hexose transport. The method includes the steps of obtaining a cell that can conduct hexose transport, and contacting the cell with an agent that specifically inhibits expression or activity of Akt1, Akt2, or both. In some embodiments, the agent that specifically inhibits expression or activity of Akt1 or Akt2 and hexose transport is partially inhibited. The agent can be an siRNA specifically targeted to Akt1, Akt2, or both.

In one aspect, the invention provides a method of identifying a gene that affects glucose transport, the method involving the steps of: (a) introducing into an adipocyte an siRNA targeted against the gene using the methods provided herein; (b) culturing the cell under conditions suitable for expression of the targeted gene; and (c) assaying glucose transport in the cell, wherein a reduction in glucose transport indicates that the targeted gene affects glucose transport, to thereby identify a gene that affects glucose transport.

The invention also provides a method for identifying an insulin response modulator, involving the steps of (a) contacting a cell expressing a protein that affects glucose transport with a test compound, wherein the protein is encoded by a gene identified according to the methods provided herein, and (b) determining the ability of the test compound to modulate an activity of the protein, such that the insulin response modulator is identified.

In a related aspect, the invention provides a method for identifying an insulin response modulator, involving the steps of: (a) contacting a cell expressing a protein that affects glucose transport with a test compound, wherein the protein is encoded by a gene identified according to the methods provided herein, and (b) determining the ability of the test compound to modulate expression of the protein, such that the insulin response modulator is identified.

In various embodiments, the modulator is a positive modulator or a negative modulator. The invention further features a modulator identified by the methods of the invention.

In another aspect, the invention provides a method of regulating glucose homeostasis in a subject comprising administering to the subject an insulin response modulator of the invention. In another aspect, the invention provides a method of regulating blood glucose levels in a subject comprising administering to the subject an insulin response modulator of the invention.

Further featured is a pharmaceutical composition that comprises the modulators identified herein.

In another aspect, the invention provides a method of treating an insulin response disease or disorder comprising administering a pharmaceutical composition of the invention. In various embodiments, the disease or disorder is selected from the group consisting of Type II diabetes, insulin resistance and obesity.

A target gene is a gene targeted for RNAi-mediated gene knockdown by an siRNA (targeted siRNA) or by an siRNA derivative. One portion of an siRNA is fully complementary to a section of the mRNA of the target gene.

A control cell or control culture is a cell or culture that has not been contacted with a modified siRNA or an siRNA derivative. The control cell or culture generally contains one or more reporter genes that are expressed or one or more endogenous genes of interest for RNAi-mediated knockdown. In some embodiments of the invention, the control cell or control culture contains an siRNA targeted to a reporter gene or to an endogenous gene of interest. In some cases, the control cell or control culture contains an introduced control sequence such as an antisense strand corresponding to the antisense strand of an siRNA or modified siRNA, or which comprises a scrambled sequence of the siRNA or modified siRNA.

A test cell or test culture contains one or more reporter genes that are expressed or one or more expressed endogenous genes of interest for RNAi-mediated gene knockdown and also contains a modified siRNA or siRNA derivative targeted to a reporter gene or to an endogenous gene of interest.

Methods of introducing siRNA into adipocytes (e.g., transfection using agents such as Lipofectamine™) have generally been unsuccessful. The present method, which employs electroporation, provides a method of introducing siRNA into adipocytes, without the use of transfection agents. Thus, the present invention makes it possible to employ gene knockdown methods that employ siRNA for the study of and development of treatments related to adipocyte function, for example, glucose transport and disorders such as diabetes.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In addition, the materials, methods, and examples are illustrative only and not intended to be limiting.

Other features and advantages of the invention will be apparent from the detailed description, drawings, and from the claims.

### DESCRIPTION OF DRAWINGS

FIG. 1A is a chart showing the sequences of Cy3-labeled lamin A/C and scrambled siRNA duplexes. Lamin A/C siRNA was designed according to the mouse lamin A mRNA sequence obtained from the NCBI database (accession # BC015302) (SEQ ID NO: 19) and is unique to mouse lamin A/C according to the mouse EST database. Lamin A/C siRNA comprises a sense strand of SEQ ID NO: 1 and antisense strand of SEQ ID NO: 2. Mouse scrambled siRNA was designed as a random sequence whose sense stand does not show homology to any gene obtained in the mouse EST data base, and which comprises the two strands of SEQ ID NO: 3 and SEQ ID NO: 4.

FIG. 1B is a series of reproductions of fluorescent micrographs (labeled 1a, 1b, 2a, 2b, 3a, 3b, 4a, and 4b) showing 3T3-L1 adipocytes (day 5) that were transfected with Cy3-labeled mouse lamin A/C or scrambled siRNA duplexes by electroporation at a concentration of 80 nmoles per 5 million cells. After re-seeding for 48 hours, nuclear membrane localization of lamin A/C was visualized by immunofluorescence using antibody against mouse lamin A/C as described herein.

FIG. 2 is a bar graph showing the results of experiments in which Cy3-labeled scrambled siRNA or lamin A/C siRNA at the concentrations indicated were electroporated with differentiated 3T3-L1 adipocytes (day 5). After immunostaining with anti-lamin A/C antibody, cells with lamin A/C staining were counted. Data represent mean [symbol] SD of three independent experiments.

FIG. 3A is a schematic drawing of the Akt1 and Akt2 proteins, Akt1 and Akt2 mRNAs, and siRNA targeted sequences for Akt1 (SEQ ID NOs: 5 and 6) and for Akt2 (SEQ ID NOs: 7 and 8).

FIG. 3B is a phophorimage of Western blots of adipocyte lysates after introduction of siRNAs as indicated for 24 or 48 hours. Blots were stained for Akt1, Akt2, and Myosin IIB. Akt1 and Akt2 siRNA duplexes were designed according to mouse Akt1 and Akt2 mRNA sequences obtained from NCBI database (accession numbers are NM_{—}009652, SEQ ID NO: 20 and U22445, SEQ ID NO: 21, respectively). Sc: scramble; A1a: akt1a siRNA; A1b: akt1b siRNA; A2a: akt2a siRNA; A2b: akt2b siRNA.

FIG. 4A is a phosphorimage of Western blots of adipocyte lysates of 3T3-L1 adipocytes that were transfected with siRNAs (40 nmoles siRNA duplexes/5×10⁶ cells) by electroporation, re-seeded for 42 hours, and serum starved for 6 hours before the treatment with insulin for 15 minutes at 37° C. 50 Tg protein of total cell lysate was used for detection of Akt1 and phospho-Thr308/309 Akt. 25 Tg protein was used for detection of Akt2, nonmuscle myosin IIB and Acrp30.

FIG. 4B is a bar graph of Akt1 and Akt2 protein levels, respectively, after introduction of Akt1b, or Akt2b, or scrambled siRNA.

FIG. 4C is a bar graph of Akt threonine 308/309 phosphorylation after introduction of scrambled, Akt1b, or Akt2b siRNA followed by incubation in O, 1 nM, or 100 nM insulin. Data are presented as a mean±SD of three independent experiments.

FIG. 5A is a phosphorimage of Western blots detecting phospho-GSK3 and GSK3 after introduction of siRNA followed by incubation in O, 1 nM, or 100 nM insulin.

FIG. 5B is a phosphorimage of Western blots detecting phospho-Erk1/2 and Erk1/2 after introduction of siRNA followed by incubation in O, 1 nM, or 100 nM insulin.

FIG. 5C is a bar graph showing phosphorylation of GSK3I quantified by measurement of the ratio of intensity of phospho-GSK3I bands versus the intensity of GSK3I protein bands.

FIG. 5D is a bar graph showing phosphorylation of Erk1/2 quantified by measurement of the ratio of the intensity of phospo-Erk1/2 bands versus the intensity of Erk1/2 protein bands. Data are presented as a mean±SD of three independent experiments.

FIG. 6A is a phosphorimage of representative Western blot images for Akt protein levels, phospho-threonine 308/309 levels, and phospho-serine 21-GSK3I levels.

FIG. 6B is a bar graph of experiments showing the dose dependence of insulin-stimulated deoxyglucose uptake. Quantitative data are presented as a mean±SD of four independent experiments.

FIG. 6C is a bar graph of experiments showing inhibition of 100 nM insulin-induced glucose uptake and GSK3I phosphorylation by knockdown Akt1 and Akt2 protein levels. Quantitative data are presented as a mean±SD of three independent experiments.

FIG. 7A is a representative image for GFP-positive cells and exofacial Myc staining. The concentration of insulin used was 100 nM. Images for Myc staining and GFP signal were taken at an optical plane near the middle of cells where the Myc signal around the rim of cells was the brightest.

FIG. 7B is a Western blot showing Akt protein levels in adipocytes transfected with Myc-Glut4-EGFP and siRNAs for 48 h.

FIG. 7C is a bar graph showing the percentage of the transfected adipocytes showing a Myc-GLUT4-GFP rim on the cell surface. Data are presented as the mean±SD of three independent experiments with >200 cells counted in each experiment.

FIG. 7D is a bar graph showing the ratio of cell surface Myc signal over the total GFP signal in adipocytes expressing Myc-GLUT4-GFP. The concentration of insulin used was 100 nM. Comparison of data presented as mean±SD was performed by using the unpaired Student's t test. *, P<0.001 for difference between cells transfected with scrambled siRNA vs. cells transfected with akt1b/akt2b siRNAs. Scr, scrambled siRNA; 1b, akt1b siRNA; 2b, akt2b siRNA.

FIG. 8A is a Western blot showing amounts of Myo1c, myosin 2b and EHD2 protein in differentiated 3T3-L1 adipocytes after expression of scrambled siRNA (lane 1), siRNA 6 (lane 2) or siRNAs 6 and 2 (lane 3).

FIG. 8B is a bar graph showing quantification of the Myo1c protein shown in FIG. 8A using a scanning densitometer.

FIG. 8C is a bar graph showing insulin-stimulated [³H]2-deoxyglucose uptake in differentiated 3T3-L1 adipocytes transfected with either scrambled siRNA, siRNA 6 or siRNAs 6 and 2. Results are the average of three separate experiments. Asterisk, P<0.0004, double asterisk, P<0.0001.

### DETAILED DESCRIPTION

The invention provides an effective method of introducing nucleic acids, e.g., siRNAs, into adipocytes. The new method employs electroporation of the adipocytes under certain conditions. The method was developed using the test protein lamin A/C and has been used to investigate the role of Akt (protein kinase B; PKB). Using the new method, virtually complete inhibition of expression of a targeted protein can be achieved while other proteins are expressed normally.

This method is useful for, e.g., drug discovery such as for identification of insulin sensitizers that can be used to treat type 2 diabetes and for drugs that alleviate obesity.

Although gene-specific knockout mice provide a tool to study acute actions of signaling molecules, phenotypes observed in mice can result from unrelated changes in gene expression and developmental regulation. For example, the present invention provides methods permitting the selective inhibition of expression in adipocytes, e.g., of Akt protein kinases in intact cultured adipocytes, through the mechanism of RNA interference (RNAi) (for reviews, see Hutvagner et al., 2002, Curr. Opin. Genet. Dev. 12:225, Hannon, 2002, Nature 418:244). This powerful approach overcomes the problems encountered in mouse gene knockouts (i.e., in transgenic mice) where loss of both Akt1 and Akt2 genes is lethal.

Methods that permit the selective inhibition of genes in cultured cells, particularly primary or secondary cells are useful for determining the functions of genes to define gene targets. Such methods are also useful for developing an inventory of expected effects when expression of a specific gene is inhibited. The inventory can then be used when screening for compounds to identify those whose effects are limited to modulating expression of the specific gene. Such a system, as is described herein, is particularly useful in adipocytes, which are associated with disorders for which drugs are sought such as obesity and diabetes (e.g., type 2 diabetes).

So that the invention may be more readily understood, certain terms are defined as follows:

The term “nucleoside” refers to a molecule having a purine or pyrimidine base covalently linked to a ribose or deoxyribose sugar. Exemplary nucleosides include adenosine, guanosine, cytidine, uridine and thymidine. The term “nucleotide” refers to a nucleoside having one or more phosphate groups joined in ester linkages to the sugar moiety. Exemplary nucleotides include nucleoside monophosphates, diphosphates and triphosphates. The terms “polynucleotide” and “nucleic acid molecule” are used interchangeably herein and refer to a polymer of nucleotides joined together by a phosphodiester linkage between 5′ and 3′ carbon atoms.

The term “RNA” or “RNA molecule” or “ribonucleic acid molecule” refers generally to a polymer of ribonucleotides. The term “DNA” or “DNA molecule” or deoxyribonucleic acid molecule” refers generally to a polymer of deoxyribonucleotides. DNA and RNA molecules can be synthesized naturally (e.g., by DNA replication or transcription of DNA, respectively). RNA molecules can be post-transcriptionally modified. DNA and RNA molecules can also be chemically synthesized. DNA and RNA molecules can be single-stranded (i.e., ssRNA and ssDNA, respectively) or multi-stranded (e.g., double stranded, i.e., dsRNA and dsDNA, respectively). Based on the nature of the invention, however, the term “RNA” or “RNA molecule” or “ribonucleic acid molecule” can also refer to a polymer comprising primarily (i.e., greater than 80% or, preferably greater than 90%) ribonucleotides but optionally including at least one non-ribonucleotides molecule, for example, at least one deoxribonucleotide and/or at least one nucleotide analog.

The term “nucleotide analog”, also referred to herein as an “altered nucleotide” or “modified nucleotide” refers to a non-standard nucleotide, including non-naturally occurring ribonucleotides or deoxyribonucleotides. Preferred nucleotide analogs are modified at any position so as to alter certain chemical properties of the nucleotide yet retain the ability of the nucleotide analog to perform its intended function.

The term “RNA analog” refers to an polynucleotide (e.g., a chemically synthesized polynucleotide) having at least one altered or modified nucleotide as compared to a corresponding unaltered or unmodified RNA but retaining the same or similar nature or function as the corresponding unaltered or unmodified RNA. As discussed above, the oligonucleotides may be linked with linkages which result in a lower rate of hydrolysis of the RNA analog as compared to an RNA molecule with phosphodiester linkages. Exemplary RNA analogues include sugar- and/or backbone-modified ribonucleotides and/or deoxyribonucleotides. Such alterations or modifications can further include addition of non-nucleotide material, such as to the end(s) of the RNA or internally (at one or more nucleotides of the RNA). An RNA analog need only be sufficiently similar to natural RNA that it has the ability to mediate (mediates) RNA interference.

The term “RNA interference” or “RNAi”, as used herein, refers generally to a sequence-specific or selective process by which a target molecule (e.g., a target gene, protein or RNA) is downregulated. In specific embodiments, the process of “RNA interference” or “RNAi” features degradation of RNA molecules, e.g., RNA molecules within a cell, said degradation being triggered by an RNA agent. Degradation is catalyzed by an enzymatic, RNA-induced silencing complex (RISC). RNAi occurs in cells naturally to remove foreign RNAs (e.g., viral RNAs). Natural RNAi proceeds via fragments cleaved from free dsRNA which direct the degradative mechanism to other similar RNA sequences. Alternatively, RNAi can be initiated by the hand of man, for example, to silence the expression of target genes.

As used herein, the term “small interfering RNA” (“siRNA”) (also referred to in the art as “short interfering RNAs”) refers to an RNA (or RNA analog) comprising between about 10-50 nucleotides (or nucleotide analogs) which is capable of directing or mediating RNA interference. An “siRNA”, having a “sequence sufficiently complementary” to a target mRNA sequence to direct or mediate RNAi means that the siRNA has a sequence sufficient to trigger the destruction of the target mRNA by the RNAi machinery (e.g., the RISC complex) or process.

The term “shRNA” or “short hairpin RNA”, as used herein, refers to an RNA agent having a stem-loop structure, comprising a first and second region of complementary sequence, the degree of complementarity and orientation of the regions being sufficient such that base pairing occurs between the regions, the first and second regions being joined by a loop region, the loop resulting from a lack of base pairing between nucleotides (or nucleotide analogs) within the loop region.

A “mRNA” or “messenger RNA” is single-stranded RNA that specifies the amino acid sequence of one or more polypeptide chains. This information is translated during protein synthesis when ribosomes bind to the mRNA.

A gene “involved” in a disorder includes a gene, the normal or aberrant expression or function of which effects or causes a disease or disorder or at least one symptom of said disease or disorder

Various methodologies of the instant invention include steps that involve comparing a value, level, feature, characteristic, property, etc. to a “suitable control”, referred to interchangeably herein as an “appropriate control”. A “suitable control” or “appropriate control” is any control or standard familiar to one of ordinary skill in the art useful for comparison purposes. In one embodiment, a “suitable control” or “appropriate control” is a value, level, feature, characteristic, property, etc. determined prior to performing an RNAi methodology, as described herein. For example, a transcription rate, mRNA level, translation rate, protein level, biological activity, cellular characteristic or property, genotype, phenotype, etc. can be determined prior to introducing a siRNA of the invention into a cell or organism. In another embodiment, a “suitable control” or “appropriate control” is a value, level, feature, characteristic, property, etc. determined in a cell or organism, e.g., a control or normal cell or organism, exhibiting, for example, normal traits. In yet another embodiment, a “suitable control” or “appropriate control” is a predefined value, level, feature, characteristic, property, etc.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below.

I. RNAi Molecules

RNA interference (RNAi) is the mechanism of sequence-specific, post-transcriptional gene silencing initiated by double-stranded RNAs (dsRNA) homologous to the gene being suppressed. dsRNAs are processed by Dicer, a cellular ribonuclease III, to generate duplexes of about 21 nt with 3′-overhangs (small interfering RNA, siRNA) which mediate sequence-specific mRNA degradation. In mammalian cells siRNA molecules are capable of specifically silencing gene expression without induction of the unspecific interferon response pathway. Thus, siRNAs have become a new and powerful alternative to other genetic tools such as antisense oligonucleotides and ribozymes to analyze loss-of-function phenotypes. Application of siRNA duplexes to interfere with the expression of a specific gene requires knowledge of target accessibility, highly effective delivery of siRNAs into target cells and for some applications long-term siRNA expression. The present invention provides an effective strategy to deliver siRNAs to target cells in cell culture, e.g., adipocytes, by the transduction of short duplex siRNAs molecules into cells, e.g., adipocyte, by using electroporation methods, as described herein. The methods of the present invention are also useful to introduce into cells, e.g., adipocytes, expression cassettes that allow transcription of functional siRNAs or their precursors by various Pol III promoters (Scherr M, Morgan M A, Eder M. Curr Med Chem. 2003 February;10(3):245-56), thereby providing endogenous expression of siRNAs.

RNAi is a remarkably efficient process whereby double-stranded RNA including for example, siRNAs and/or long ds RNA, induces the sequence-specific degradation of targeted mRNA in animals and plant cells (Hutvagner and Zamore, Curr. Opin. Genet. Dev.:12, 225-232 (2002); Sharp, Genes Dev., 15:485-490 (2001)). In mammalian cells, RNAi can be triggered by 21-nucleotide (nt) duplexes of small interfering RNA (siRNA) (Chiu et al., Mol. Cell. 10:549-561 (2002); Elbashir et al., Nature 411:494-498 (2001)), or by micro-RNAs (mRNA), functional small-hairpin RNA (shRNA), or other dsRNAs which can be expressed in vivo using DNA templates with RNA polymerase III promoters (Zeng et al., Mol. Cell 9:1327-1333 (2002); Paddison et al., Genes Dev. 16:948-958 (2002); Lee et al., Nature Biotechnol. 20:500-505 (2002); Paul et al., Nature Biotechnol. 20:505-508 (2002); Tuschl, T., Nature Biotechnol. 20:440-448 (2002); Yu et al., Proc. Natl. Acad. Sci. USA 99(9):6047-6052 (2002); McManus et al., RNA 8:842-850 (2002); Sui et al., Proc. Natl. Acad. Sci. USA 99(6):5515-5520 (2002).)

Accordingly, the invention provides nucleic acid molecules (i.e., RNAi molecules) that are targeted to an mRNA sequence of a gene that affects glucose transport, for example, Akt1, Akt2, Myo1c or CISK. In particular embodiments, the genes that affect glucose transport comprise the RNAi target nucleotide sequences shown in Table 1.

RNAi molecules of the invention include siRNA molecules. The siRNA constructs of the invention include dsRNA molecules comprising 16-30, e.g., 16, 17, 18, 19, 20, 21, 22, 23, 24, 25, 26, 27, 28, 29, or 30 nucleotides in each strand, wherein one of the strands is substantially identical, e.g., at least 80% (or more, e.g., 85%, 90%, 95%, or 100%) identical, e.g., having 3, 2, 1, or 0 mismatched nucleotide(s), to a target region in the mRNA and the other strand is identical or substantially identical to the first strand. An siRNA typically has a 2-3 nucleotide 3′ overhanging end, a 5′phosphate (upon extraction from a cell) and a 3′hydroxyl terminus. Methods of synthesizing RNAs and modifying RNAs are known in the art (e.g., Hwang et al., 1999, Proc. Nat. Acad. Sci. USA 96:12997-13002; and Huq and Rana, 1997, Biochem. 36:12592-12599). siRNAs can be purchased commercially (e.g., Dharmacon Research, Inc., Layfayette, Colo.; Ambion, Inc., Austin, Tex.).

The siRNA molecules of the invention can be chemically synthesized, or can be transcribed in vitro from a DNA template, or in vivo from e.g., shRNA, or, by using recombinant human DICER enzyme, to cleave in vitro transcribed dsRNA templates into pools of 20- or 21-bp duplex RNA mediating RNAi. The siRNA molecules can be designed using any method known in the art, for instance, by using the following protocol:
1. Beginning with the AUG start codon, look for AA dinucleotide sequences; each AA and the 3′ adjacent 16 or more nucleotides are potential siRNA targets (see FIGS. 15, 16, 34, 35, 36). siRNAs taken from the 5′ untranslated regions (UTRs) and regions near the start codon (within about 75 bases or so) may be less useful as they may be richer in regulatory protein binding sites, and UTR-binding proteins and/or translation initiation complexes may interfere with binding of the siRNP or RISC endonuclease complex. Thus, in one embodiment, the nucleic acid molecules are selected from a region of the cDNA sequence beginning 50 to 100 nt downstream of the start codon. Further, siRNAs with lower G/C content (35-55%) may be more active than those with G/C content higher than 55%. Thus in one embodiment, the invention includes nucleic acid molecules having 35-55% G/C content. In addition, the strands of the siRNA can be paired in such a way as to have a 3′ overhang of 1 to 4, e.g., 2, nucleotides. Thus in another embodiment, the nucleic acid molecules can have a 3′ overhang of 2 nucleotides, such as TT. The overhanging nucleotides can be either RNA or DNA.
2. Using any method known in the art, compare the potential targets to the appropriate genome database (human, mouse, rat, etc.) and eliminate from consideration any target sequences with significant homology to other coding sequences. One such method for such sequence homology searches is known as BLAST, which is available at the National Center for Biotechnology Information web site of the National Institutes of Health.
3. Select one or more sequences that meet your criteria for evaluation. Further general information about the design and use of siRNA can be found in “The siRNA User Guide,” available at the web site of the laboratory of Dr. Thomas Tuschl at Rockefeller University.

In some embodiments of the invention, an siRNA exhibits a relatively low level of toxicity. For example, a concentration of an siRNA that inhibits expression of a targeted sequence has relatively low toxicity when at least 50% of the cells in a culture treated with the siRNA are viable when expression of the targeted sequence is decreased by 50% compared to expression in a cell that is not treated with the siRNA. Low toxicity may be associated with greater cell viability, e.g., at least 60%, 75%, 85%, 90%, 95%, or 100%. Methods of measuring cell viability are known in the art and include trypan blue exclusion. This is, for example, in contrast to a knockout of a targeted gene (such as in a transgenic animal). Therefore, an advantage of the invention is that it permits manipulation (i.e., inhibition) of expression of a gene that can be lethal to the a cell or an animal when expression of the gene is manipulated by genetically engineering the gene.

The RNAi agents of the present invention can also include small hairpin RNAs (shRNAs), and expression constructs engineered to express shRNAs. Transcription of shRNAs is initiated at a polymerase III (pol III) promoter, and is thought to be terminated at position 2 of a 4-5-thymine transcription termination site. Upon expression, shRNAs are thought to fold into a stem-loop structure with 3′ UU-overhangs; subsequently, the ends of these shRNAs are processed, converting the shRNAs into siRNA-like molecules of about 21 nucleotides. Brummelkamp et al., Science 296:550-553 (2002); Lee et al, (2002). supra; Miyagishi and Taira, Nature Biotechnol. 20:497-500 (2002); Paddison et al. (2002), supra; Paul (2002), supra; Sui (2002) supra; Yu et al. (2002), supra. More information about shRNA design and use can be found on the internet at the following addresses:
katahdin.cshl.org:9331/RNAi/docs/BseRI-BamHI_Strategy.pdf and
katahdin.cshl.org:933 l/RNAi/docs/Web_version_of_PCR_strategy1.pdf.

Expression constructs of the present invention include any construct suitable for use in the appropriate expression system and include, but are not limited to, retroviral vectors, linear expression cassettes, plasmids and viral or virally-derived vectors, as known in the art. Such expression constructs can include one or more inducible promoters, RNA Pol III promoter systems such as U6 snRNA promoters or H1 RNA polymerase III promoters, or other promoters known in the art. The constructs can include one or both strands of the siRNA. Expression constructs expressing both strands can also include loop structures linking both strands, or each strand can be separately transcribed from separate promoters within the same construct. Each strand can also be transcribed from a separate expression construct. Tuschl (2002), supra).

Methods for expressing siRNA duplexes within cells from recombinant DNA constructs to allow longer-term target gene suppression in cells are known in the art, including mammalian Pol III promoter systems (e.g., H1 or U6/snRNA promoter systems (Tuschl (2002), supra) capable of expressing functional double-stranded siRNAs; (Bagella et al., J. Cell. Physiol. 177:206-213 (1998); Lee et al. (2002), supra; Miyagishi et al. (2002), supra; Paul et al. (2002), supra; Yu et al. (2002), supra; Sui et al. (2002), supra). Transcriptional termination by RNA Pol III occurs at runs of four consecutive T residues in the DNA template, providing a mechanism to end the siRNA transcript at a specific sequence. The siRNA is complementary to the sequence of the target gene in 5′-3′ and 3′-5′ orientations, and the two strands of the siRNA can be expressed in the same construct or in separate constructs. Hairpin siRNAs, driven by H1 or U6 snRNA promoter and expressed in cells, can inhibit target gene expression (Bagella et al. (1998), supra; Lee et al. (2002), supra; Miyagishi et al. (2002), supra; Paul et al. (2002), supra; Yu et al. (2002), supra; Sui et al. (2002) supra). Constructs containing siRNA sequence under the control of T7 promoter also make functional siRNAs when cotransfected into the cells with a vector expressing T7 RNA polymerase (Jacque (2002), supra). A single construct may contain multiple sequences coding for siRNAs, such as multiple regions of genes encoding, e.g, Akt1, Akt2, CISK or Myo1c, targeting the same gene or multiple genes, and can be driven, for example, by separate Pol III promoter sites.

Animal cells express a range of noncoding RNAs of approximately 22 nucleotides termed micro RNA (miRNAs) which can regulate gene expression at the post transcriptional or translational level during animal development. One common feature of miRNAs is that they are all excised from an approximately 70 nucleotide precursor RNA stem-loop, probably by Dicer, an RNase III-type enzyme, or a homolog thereof. By substituting the stem sequences of the miRNA precursor with miRNA sequence complementary to the target mRNA, a vector construct that expresses the novel miRNA can be used to produce siRNAs to initiate RNAi against specific mRNA targets in mammalian cells (Zeng (2002), supra). When expressed by DNA vectors containing polymerase III promoters, micro-RNA designed hairpins can silence gene expression (McManus (2002), supra). In an animal model, whole-embryo electroporation can efficiently deliver synthetic siRNA into post-implantation mouse embryos (Calegari et al., Proc. Natl. Acad. Sci. USA 99(22):14236-40 (2002)). In adult mice, efficient delivery of siRNA can be accomplished by “high-pressure” delivery technique, a rapid injection (within 5 seconds) of a large volume of siRNA containing solution into animal via the tail vein (Liu (1999), supra; McCaffrey (2002), supra; Lewis, Nature Genetics 32:107-108 (2002)). Nanoparticles and liposomes can also be used to deliver siRNA into animals.

Negative control siRNAs should have the same nucleotide composition as the selected siRNA, but without significant sequence complementarity to the appropriate genome. Such negative controls can be designed by randomly scrambling the nucleotide sequence of the selected siRNA; a homology search can be performed to ensure that the negative control lacks homology to any other gene in the appropriate genome. In addition, negative control siRNAs can be designed by introducing one or more base mismatches into the sequence.

The nucleic acid compositions of the invention include both unmodified siRNAs and modified siRNAs as known in the art, such as crosslinked siRNA derivatives. Crosslinking can be employed to alter the pharmacokinetics of the composition, for example, to increase half-life in the body. Thus, the invention includes siRNA derivatives that include siRNA having two complementary strands of nucleic acid, such that the two strands are crosslinked. For example, a 3′ OH terminus of one of the strands can be modified, or the two strands can be crosslinked and modified at the 3′OH terminus. The siRNA derivative can contain a single crosslink (e.g., a psoralen crosslink). Modifying siRNA derivatives may improve cellular uptake or enhance cellular targeting activities of the resulting siRNA derivative as compared to the corresponding siRNA, are useful for tracing the siRNA derivative in the cell, or improve the stability of the siRNA derivative compared to the corresponding siRNA.

The nucleic acid compositions of the invention can be unconjugated or can be conjugated to another moiety, such as a nanoparticle, to enhance a property of the compositions, e.g., a pharmacokinetic parameter such as absorption, efficacy, bioavailability, and/or half-life. The conjugation can be accomplished by methods known in the art, e.g., using the methods of Lambert et al., Drug Deliv. Rev.:47(1), 99-112 (2001) (describes nucleic acids loaded to polyalkylcyanoacrylate (PACA) nanoparticles); Fattal et al., J. Control Release 53(1-3):137-43 (1998) (describes nucleic acids bound to nanoparticles); Schwab et al., Ann. Oncol. 5 Suppl. 4:55-8 (1994) (describes nucleic acids linked to intercalating agents, hydrophobic groups, polycations or PACA nanoparticles); and Godard et al., Eur. J. Biochem. 232(2):404-10 (1995) (describes nucleic acids linked to nanoparticles).

The nucleic acid molecules of the present invention can also be labeled using any method known in the art; for instance, the nucleic acid compositions can be labeled with a fluorophore, e.g., Cy3, fluorescein, or rhodamine. The labeling can be carried out using a kit, e.g., the SILENCER™ siRNA labeling kit (Ambion). Additionally, the siRNA can be radiolabeled, e.g., using ³H, ³²P, or other appropriate isotope.

Engineered RNA precursors, introduced into cells (e.g., adipocytes) or whole organisms as described herein, will lead to the production of a desired siRNA molecule. Such an siRNA molecule will then associate with endogenous protein components of the RNAi pathway to bind to and target a specific mRNA sequence for cleavage and destruction. In this fashion, the mRNA to be targeted by the siRNA generated from the engineered RNA precursor will be depleted from the cell or organism, leading to a decrease in the concentration of the protein encoded by that mRNA in the cell or organism. The RNA precursors are typically nucleic acid molecules that individually encode either one strand of a dsRNA or encode the entire nucleotide sequence of an RNA hairpin loop structure.

Moreover, because RNAi is believed to progress via at least one single stranded RNA intermediate, the skilled artisan will appreciate that ss-siRNAs (e.g., the antisense strand of a ds-siRNA) can also be designed (e.g., for chemical synthesis) generated (e.g., enzymatically generated) or expressed (e.g., from a vector or plasmid) as described herein and utilized according to the claimed methodologies. Moreover, in invertebrates, RNAi can be triggered effectively by long dsRNAs (e.g., dsRNAs about 100-1000 nucleotides in length, preferably about 200-500, for example, about 250, 300, 350, 400 or 450 nucleotides in length) acting as effectors of RNAi. (Brondani et al., Proc Natl Acad Sci USA. 2001 Dec. 4;98(25):14428-33. Epub 2001 Nov. 27).

The siRNA molecules of the present invention can target the following sequences of the target gene, e.g., the siRNA molecules may comprise, as one of their strands, an RNA sequence corresponding to any one of the following DNA sequences (e.g., the sense strand of the siRNA duplex) and the corresponding sequences of allelic variants thereof. Sequences in the table are represented as target gene sequences (i.e., cDNA sequences). The skilled artisan will appreciate, however, that siRNA strands, e.g., sense strands, comprise corresponding ribonucleotides, and that antisense strands comprise complementary ribonucleotide sequences. Additional deoxythymidine overhangs are also contemplated as described herein.

TABLE 1
siRNA CANDIDATE TARGET SEOUENCES

| | siRNA CANDIDATE | |
|---|---|---|
| GENES | TARGET SEQUENCES | SEQUENCES |
| Aktl | ACGACGTAGCCATTGTGAA | SEQ ID NO:27 |
| Mus | CGACGTAGCCATTGTGAAG | SEQ ID NO:28 |
| musculus | CTTCCTCCTCAAGAACGAT | SEQ ID NO:29 |
| | GGCAGGAAGAAGAGACGAT | SEQ ID NO:30 |
| | GACGATGGACTTCCGATCA | SEQ ID NO:31 |
| | AGCACCGTGTGACCATGAA | SEQ ID NO:32 |
| | CTACTTGCACTCCGAGAAG | SEQ ID NO:33 |
| | GGATGGTGCCACTATGAAG | SEQ ID NO:34 |
| | TGGTGCCACTATGAAGACA | SEQ ID NO:35 |
| | GGATGCCAAGGAGATCATG | SEQ ID NO:36 |
| | CCGGTTCTTTGCCAACATC | SEQ ID NO:37 |
| | CTGACACCAGGTATTTCGA | SEQ ID NO:38 |
| | CACCAGGTATTTCGATGAG | SEQ ID NO:39 |
| | GGTATTTCGATGAGGAGTT | SEQ ID NO:40 |
| | TTTCGATGAGGAGTTCACA | SEQ ID NO:41 |
| Akt2 | AACGTGGTGAATACATCAA | SEQ ID NO:42 |
| Mus | ACGTGGTGAATACATCAAG | SEQ ID NO:43 |
| musculus | CGTGGTGAATACATCAAGA | SEQ ID NO:44 |
| | CCATGAATGACTTCGATTA | SEQ ID NO:45 |
| | GGAGGTCATCATTGCAAAG | SEQ ID NO:46 |
| | GTATTTGCACTCGAGAGAT | SEQ ID NO:47 |
| | CTCGAGAGATGTGGTGTAC | SEQ ID NO:48 |
| | CCGTGACATCAAGCTGGAA | SEQ ID NO:49 |
| | ACCTTATGTTGGACAAAGA | SEQ ID NO:50 |
| | GGTCATGGAGCATAGATTC | SEQ ID NO:51 |
| | CACAAGGTACTTTGATGAC | SEQ ID NO:52 |
| | TGAGCGACGTGGCTATTGT | SEQ ID NO:53 |
| Akt1 | CTGTCATCGAACGCACCTT | SEQ ID NO:54 |
| Homo | TCGAACGCACCTTCCATGT | SEQ ID NO:55 |
| Sapiens | TGAACGAGTTTGAGTACCT | SEQ ID NO:56 |
| | ACGAGTTTGAGTACCTGAA | SEQ ID NO:57 |
| | TGGCGCTGAGATTGTGTCA | SEQ ID NO:58 |
| | CCAGATGCAACCTCACTAT | SEQ ID NO:59 |
| | GATGCAACCTCACTATGGT | SEQ ID NO:60 |
| | TGATCTCTCCACGGTAGCA | SEQ ID NO:61 |
| Akt2 | CAAGCGTGGTGAATACATC | SEQ ID NO:62 |
| Homo | AGCGTGGTGAATACATCAA | SEQ ID NO:63 |
| Sapiens | GCGTGGTGAATACATCAAG | SEQ ID NO:64 |
| | CAGTCATCGAGAGGACCTT | SEQ ID NO:65 |
| | CTTCGATGATGAATTTACC | SEQ ID NO:66 |
| | TGGAGCACAGGTTCTTCCT | SEQ ID NO:67 |
| CISK | CTCAATTTCTGTCCTTCAA | SEQ ID NO:68 |
| Mus | AAGAAGAGGTTCACGGTTT | SEQ ID NO:69 |
| Musculus | AGAAGAGGTTCACGGTTTA | SEQ ID NO:70 |
| | GAAGAGGTTCACGGTTTAT | SEQ ID NO:71 |
| | AGAGGTTCACGGTTTATAA | SEQ ID NO:72 |
| | GAGGTTCACGGTTTATAAA | SEQ ID NO:73 |
| | GGTTCACGGTTTATAAAGT | SEQ ID NO:74 |
| | GAAGCGAGTGGTTTGTCTT | SEQ ID NO:75 |
| | GAACTTGGTCAGATATCCA | SEQ ID NO:76 |
| | GATATCCAGAGCTTTACAA | SEQ ID NO:77 |
| | TCCAGATGTCCGAGCATTC | SEQ ID NO:78 |
| | GTACTTCGAAGCCACATTC | SEQ ID NO:79 |
| | AATCCTCATGCTAAACCAA | SEQ ID NO:80 |
| | AACCAACTGACTTCGATTT | SEQ ID NO:81 |
| | AACGGAAACTGGATGGAAA | SEQ ID NO:82 |
| | TATTATGGCTGAACGCAAT | SEQ ID NO:83 |
| | AAGAAGGAATCGCTATTTC | SEQ ID NO:84 |
| | TGACAATATTCTTCACAAG | SEQ ID NO:85 |
| | TCGTGAATGCCAGTGTTCT | SEQ ID NO:86 |
| Myo1C | CCGTGGTGTCAGTTTCTAT | SEQ ID NO:87 |
| Mus | TGAAGTACCACCTCATTTG | SEQ ID NO:88 |
| Musculus | AGTACCACCTCATTTGTTT | SEQ ID NO:89 |
| | GTACCACCTCATTTGTTTG | SEQ ID NO:90 |
| | AGACTCTCCGCAACGATAA | SEQ ID NO:91 |
| | GACTCTCCGCAACGATAAC | SEQ ID NO:92 |
| | CTCTCCGCAACGATAACTC | SEQ ID NO:93 |
| | AATCACGGAGAGCGGAACT | SEQ ID NO:94 |
| | ATCACGGAGAGCGGAACTT | SEQ ID NO:95 |
| | GCTACTTGTACCTGGTGAA | SEQ ID NO:96 |
| | ACGACAAGAGTGACTGGAA | SEQ ID NO:97 |
| | AGAGTGACTGGAAGGTTAT | SEQ ID NO:98 |
| | GAGTGACTGGAAGGTTATG | SEQ ID NO:99 |
| | GTGACTGGAAGGTTATGAG | SEQ ID NO:100 |
| | GTTCCGCCTTCTGCATTAT | SEQ ID NO:101 |
| | CAGGAGGATTGGATTTCTT | SEQ ID NO:102 |
| | CTTAGGAGCAATAGAGAGA | SEQ ID NO:103 |
| | CTGCTGACACTTCTGCAAT | SEQ ID NO:104 |
| | GGTGACCTACAGTGTGACT | SEQ ID NO:105 |
| | TCCGACATCAGGTGAAGTA | SEQ ID NO:106 |
| | CTAAGATCTTCATCCGATT | SEQ ID NO:107 |
| | AGGCGGTGGCTAGTGAAAT | SEQ ID NO:108 |
| | GGCGGTGGCTAGTGAAATT | SEQ ID NO:109 |
| | AGCAGAGAATTGATTATGC | SEQ ID NO:110 |
| | ATTGATTATGCCAACCTAA | SEQ ID NO:111 |
| | TTGATTATGCCAACCTAAC | SEQ ID NO:112 |
| | TGCCAACCTAACCGGAATC | SEQ ID NO:113 |
| | ACCTAACCGGAATCTCTGT | SEQ ID NO:114 |
| | TCATGTGATCGAGACACTA | SEQ ID NO:115 |
| | TGTGATCGAGACACTAACC | SEQ ID NO:116 |
| | TCGAGACACTAACCAAGAC | SEQ ID NO:117 |
| | CCGCGTGAACAATATCAAC | SEQ ID NO:118 |
| | CGGCATCATTGACTTCACA | SEQ ID NO:119 |
| | GCACATCTCACTGCCTTTC | SEQ ID NO:120 |
| | TGCCTTAGGATGACAACCT | SEQ ID NO:121 |
| Myo1C | GATCTACAGCCGGCAACAT | SEQ ID NO:122 |
| Homo | TCTACAGCCGGCAACATAT | SEQ ID NO:123 |
| Sapiens | ACGACAAGAGTGACTGGAA | SEQ ID NO:124 |
| | AGTCGGAGCAGGAGGAATA | SEQ ID NO:125 |
| | ATTCCGCCTTCTGCACTAT | SEQ ID NO:126 |
| | TTCCGCCTTCTGCACTATG | SEQ ID NO:127 |
| | ACCTTAAGGAGACCATGTG | SEQ ID NO:128 |
| | CCTTAAGGAGACCATGTGT | SEQ ID NO:129 |
| | CCATGTGTAGCTCAAAGAA | SEQ ID NO:130 |
| | GCGAGCTCAGTGACAAGAA | SEQ ID NO:131 |
| | TCGCCGCAAATACGAAGCT | SEQ ID NO:132 |
| | ATACGAAGCTTTCCTGCAA | SEQ ID NO:133 |
| | TACGAAGCTTTCCTGCAAA | SEQ ID NO:134 |
| | CTCGGCTTGGTACAGATGA | SEQ ID NO:135 |
| | GGATTGATTACGCCAACCT | SEQ ID NO:136 |
| | GCGTGCGGACATAAAGCAA | SEQ ID NO:137 |
| | TTGAGACGCTGACCAAGAC | SEQ ID NO:138 |
| | ACCGCGTGAACAGCATCAA | SEQ ID NO:139 |
| | GTTACCAAAGAGTCGAATT | SEQ ID NO:140 |
| | AGAGTCGAATTTCCAGACA | SEQ ID NO:141 |

II. Adipocyte Cultures

The invention relates to introduction of nucleic acids such as siRNAs into adipocytes. Adipocytes can be cultured using methods known in the art (for example, see Adipose Tissue Protocols, Methods in Molecular Biology, v. 155, ed. Gérard Ailhaud. Totowa, N.J., Humana Press, 2001). Example 1 (infra) describes one such method in which 3T3-L1 cells are differentiated into adipocytes. Other methods that can be used include differentiation of mesenchymal cells into adipocytes (e.g., using reagents such as those available from StemCell Technologies, Inc., Vancouver, B.C.), using adipocytes from dissected adipose tissue (typically after treatment to dissociate the cells), and commercially available methods for generating adipocytes (e.g., Adipocyte Differentiation Kit, Stratagene, Inc.; PromoCell, Heidelberg, Germany).

To prepare cells for introduction of nucleic acid using the methods described herein, adipocytes are detached from their culture substrate or dissociated using standard methods.

III. Introduction of Nucleic Acid Molecules into Adipocytes

In general, ribonucleic acids, deoxynucleic acids, and variants of these can be introduced into adipocytes. The nucleic acids can be nucleic acid vectors, e.g., that contain a sequence encoding a gene product or fragment of a gene product that is to be expressed in an adipocyte, antisense RNAs, siRNAs, siRNA precursors or vectors that express siRNA precursors. The nucleic acids can be double- or single-stranded.

Nucleic acid sequence that is to be introduced into an adipocyte is between about 2-10,000 bases in length, for example between about 2-5,000 bases in length, 2-1000 bases in length, 2-500 bases in length, 2-300 bases, 2-200 bases, 2-100 bases, 2-50 bases, or 2-25 bases or 1-15 bases. When the nucleic acid is a siRNA, each strand is generally between about 16-30 nucleotides in length, e.g., 16, 17, 18, 19, 20, 21, 22, 23, 24, 25, 26.27, 28, 29, or 30 nucleotides in length. Methods of preparing such nucleic acids are known in the art. Typically a nucleic acid sample that is to be used in the methods described herein is desalted before being introduced into a cell.

In general, a nucleic acid molecule is introduced into a cell after washing in a suitable buffer, e.g., in phosphate-buffered saline (PBS), and then resuspended in a suitable buffer, e.g., PBS. Other suitable buffers include, without limitation, Krebs/Ringer/HEPES buffer. The resuspended cells are mixed with nucleic acid resuspended in PBS or other buffers suitable for electroporation procedures. For example, 0.1-80 nmoles, typically about 20 nM siRNA are used. About 1-10 million cells can be used in the methods; typically, about 5 million cells are used in each procedure. Typically the cells are electroporated at room temperature although other temperatures may be used. The mixture is then exposed to a pulse of electroporation with an appropriate apparatus (e.g., a Bio-Rad Gene Pulser II system; Bio-Rad Laboratories, Hercules, Calif.) at a setting which allows efficient introducing of siRNA into the cells, e.g., at a setting of between about 0.001-20 kV, about 0.01-2.0 kV, about 0.02-1.0 kV, about 0.05-0.5 kV, about 0.1-0.25 kV, and preferably at a setting of about 0.11, 0.12, 0.13, 0.14, 0.15, 0.16, 0.17, 0.18, 0.19, 0.20, 0.21, 0.22, 0.23, 0.24 or 0.25 kV; and at a setting of between about 10-5000 μF, about 100-3000 μF, about 200-1800 μF, about 350-1550 μF, about 5-1350 μF, about 750-1150 μF, about 850-1050 μF, about 900-1000 μF, and preferably at a setting of about 900, 910, 920, 930, 940, 950, 960, 970, 980, 990, or 100 μF capacitance. In a preferred embodiment, the mixture is exposed to a pulse of electroporation at a setting of 0.18 kV and 960 μF. About 400-600 μl of cells is typically used in the procedure, e.g., 450 μl of cells. After electroporation, the cells are immediately mixed with fresh medium and incubated at 37° C. for, e.g., 10 minutes, before reseeding onto multiple-well plates that are appropriate for subsequent uses such as deoxyglucose uptake assays, Western blotting, or immunofluorescence microscopic analysis. Analyses are carried out at an appropriate time after reseeding, e.g., at about 2, 4, 8, 12, 16, 20, 24, 30, 36, 42, 48, 54, 60, 72, 78, 94, or up to about 120 hours after reseeding. In a preferred embodiment, analyses are carried out at 24 to 48 hours after reseeding.

An siRNA, introduced into cells or whole organisms as described herein, will associate with endogenous protein components of the RNAi pathway to bind to and target a specific mRNA sequence for cleavage and destruction. In this fashion, the mRNA targeted by the siRNA is depleted from the cell or organism, leading to a decrease in the concentration of the protein encoded by that mRNA in the cell or organism.

The method is useful, for example, if one is seeking to discover a small molecule that reduces the activity of a nucleic acid sequence whose expression (or overexpression) leads to abnormal glucose metabolism. A key question is whether or not decreasing the expression or activity of this nucleic acid sequence would have unexpected effects, particularly deleterious effects, on a cell. By expressing an siRNA derivative that targets for destruction by the RNAi pathway the mRNA encoding the nucleic acid sequence in an adipocyte, the deleterious effects of such a potential drug can be determined. That is, the methods described herein allows rapid assessment of the suitability of the nucleic acid sequence and its product as a drug target.

RNAi provides a new approach for elucidation of gene function. RNAi-mediated gene knockdown is useful for genome-wide analysis of gene function as well as target validation of potentially therapeutic genes. siRNAs are a useful tool for cell biologists studying mammalian gene function. For example, siRNAs are useful for the analysis of general cell biological mechanisms such as mitosis, processing and trafficking of RNA transcripts, the formation of cellular junctions, and membrane trafficking. Reagents that can be used for such analyses (e.g. siRNAs that have increased stability in a cell compared to their corresponding, unmodified siRNA) have commercial value for use in such research.

A selected gene can be knocked down by use of an siRNA and the resultant phenotype can be observed. However, knockdown of an essential gene could be lethal or toxic and may affect many pathways in the cell. Therefore, in some cases it is desirable to provide to the cell an siRNA that is not maximally efficient at knockdown (i.e., inhibiting expression of the protein translated from the targeted sequence). The adverse effects of an overly efficient knockdown can be modulated by contacting the cell with an siRNA derivative that has reduced RNAi activity compared to a corresponding siRNA. An siRNA derivative is a modified siRNA. Modifications include, without limitation, crosslinking or blocking of a 3′terminus (Chiu and Rana, 2002, Mol Cell 10:549-561). Suitable concentrations of an siRNA derivative used for this purpose include concentrations that do not maximally inhibit RNAi activity and ameliorate the undesirable effect of the siRNA. An amount of an siRNA derivative that can cause knockdown with less efficiency than a corresponding siRNA can be determined using methods known in the art. For example, a comparison is made between the level of expression in a cell transfected with an siRNA targeted to a specific nucleic acid sequence and the level of expression of the specific nucleic acid sequence in a cell that was transfected with a derivative of the siRNA. A higher level of expression in the cell transfected with the siRNA derivative (and lower level of expression than in an untransfected or mock transfected control cell) indicates that the siRNA is less efficient at knockdown of expression. Such derivatives can be useful for knockdown experiments in which more efficient knockdown is lethal to the cell. Therefore, in some cases, a useful siRNA derivative is one that inhibits RNAi by less than 100%. For example, an siRNA derivative that is useful for reducing the RNAi effect of an siRNA can inhibit RNAi activity by less than, e.g., 90%, 75%, 50%, 25%, or 10%.

IV. Nucleic Acid Targets

The nucleic acid targets of siRNAs as described herein may be any gene associated with Diabetes II, insulin resistance or obesity, or which is important for glucose transport or insulin responsiveness, including but not limited to, eg., a gene encoding Akt1, Akt2, CISK, or Myo1c. In particular embodiments, the target genes comprise the target nucleotide sequences shown in Table 1.

Accordingly, the mRNA sequence of Akt1 can be any ortholog of Akt1, such as sequences substantially identical to the S. cerevisiae, C. elegans, D. melanogaster, mouse, or human Akt1, including but not limited to GenBank Accession Nos. NM_{—}009652 (GI:6753033;Mus musculus) (SEQ ID NO:20); NM_{—}005163 (GI:4885060; Homo sapiens) (SEQ ID NO: 22). The mRNA sequence of Akt2 can be any ortholog of Akt2, such as sequences substantially identical to the S. cerevisiae, C. elegans, D. melanogaster, mouse or human Akt2, including but not limited to GenBank Accession Nos. U22445 (GI:942577; Mus musculus) (SEQ ID NO:21); NM_{—}001626 (GI:6715585; Homo sapiens) (SEQ ID NO:23). The mRNA sequence of CISK can be any ortholog of CISK, such as sequences substantially identical to the S. cerevisiae, C. elegans, D. melanogaster, human or mouse CISK, including but not limited to GenBank Accession No. AF312007 (GI:11321320; Mus musculus) (SEQ ID NO:24); GenBank Accession No. NM_{—}013257.3 (GI:25168264; Homo sapiens, variant 1); GenBank Accession No. NM_{—}170709 (GI:25168266; Homo sapiens, variant 2). The mRNA sequence of Myo1c can be any ortholog of Myo1c, such as sequences substantially identical to the S. cerevisiae, C. elegans, D. melanogaster, human or mouse Myo1c, including but not limited to GenBank Accession Nos. NM_{—}008659 (GI:31543277; Mus musculus) (SEQ ID NO:25); NM_{—}033375 (GI:24415399; Homo sapiens) (SEQ ID NO:26).

The term “ortholog” as used herein refers to a sequence which is substantially identical to a reference sequence. The term “substantially identical” is used herein to refer to a first amino acid or nucleotide sequence that contains a sufficient or minimum number of identical or equivalent (e.g., with a similar side chain) amino acid residues or nucleotides to a second amino acid or nucleotide sequence such that the first and second amino acid or nucleotide sequences have a common structural domain or common functional activity. For example, amino acid or nucleotide sequences that contain a common structural domain having at least about 60%, or 65% identity, likely 75% identity, more likely 85%, 90%. 91%, 92%, 93%, 94%, 95%, 96%, 97%, 98% or 99% identity are defined herein as substantially identical.

Calculations of homology or sequence identity between sequences (the terms are used interchangeably herein) are performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In one embodiment, the length of a reference sequence aligned for comparison purposes is at least 50%, at least 60%, at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In one embodiment, the percent identity between two amino acid sequences is determined using the Needleman and Wunsch (J. Mol. Biol. 48:444-453 (1970)) algorithm which has been incorporated into the GAP program in the GCG software package (available at the official Accelrys web site), using either a Blossum 62 matrix or a PAM250 matrix, and a gap weight of 16, 14, 12, 10, 8, 6, or 4 and a length weight of 1, 2, 3, 4, 5, or 6. In yet another embodiment, the percent identity between two nucleotide sequences is determined using the GAP program in the GCG software package (available at the official Accelrys web site), using a NWSgapdna.CMP matrix and a gap weight of 40, 50, 60, 70, or 80 and a length weight of 1, 2, 3, 4, 5, or 6. One set of parameters (and the one that can be used if the practitioner is uncertain about what parameters should be applied to determine if a molecule is within a sequence identity or homology limitation of the invention) are a Blossum 62 scoring matrix with a gap penalty of 12, a gap extend penalty of 4, and a frameshift gap penalty of 5.

The percent identity between two amino acid or nucleotide sequences can be determined using the algorithm of E. Meyers and W. Miller (CABIOS, 4:11-17 (1989)) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other orthologs, e.g., family members or related sequences. Such searches can be performed using the NBLAST and XBLAST programs (version 2.0) of Altschul, et al. J. Mol. Biol. 215:403-10 (1990). BLAST nucleotide searches can be performed with the NBLAST program, score=100, wordlength=12 to obtain nucleotide sequences homologous to known TEF nucleic acid molecules of the invention. BLAST protein searches can be performed with the XBLAST program, score=50, wordlength=3 to obtain amino acid sequences homologous to known TEF protein molecules. To obtain gapped alignments for comparison purposes, Gapped BLAST can be utilized as described in Altschul et al., Nucleic Acids Res. 25:3389-3402 (1997). When utilizing BLAST and Gapped BLAST programs, the default parameters of the respective programs (e.g., XBLAST and NBLAST) can be used. See the National Center for Biotechnology Information web site of the National Institutes of Health.

Orthologs can also be identified using any other routine method known in the art, such as screening a cDNA library, e.g., a human cDNA library, using a probe designed to identify sequences which are substantially identical to a reference sequence.

V. Functional Assays

Functional assays are used to determine the effects of knockdown of a specific gene, e.g., after depletion of a targeted sequence using siRNA methodology in adipocytes. Such assays are particularly useful for determining the effects of specific gene knockdown of nucleic acid sequences involved in glucose transport or implicated as effectors in diabetes. Such assays are known in the art and some are described herein, e.g., as described in Example 1. Such assays include, without limitation, assaying deoxyglucose uptake or MYC-tagged Glut4 for a glucose translocation assay.

A. Cell Based Assays

In one embodiment, an assay is a cell-based assay in which a cell capable of expressing a polypeptide encoded by a gene involved in glucose transport, or biologically active portion thereof, is contacted with a test compound and the ability of the test compound to modulate the expression of the polypeptide, or biologically active portion thereof, is determined. In another embodiment, an assay is a cell-based assay in which a cell which expresses a polypeptide encoded by a gene involved in glucose transport (or biologically active portions thereof) is contacted with a test compound and the ability of the test compound to modulate the activity of the polypeptide (or biologically active portions thereof) is determined. The cell, for example, can be of mammalian origin or a yeast cell. In a preferred embodiment, the cell is an adipocyte. The polypeptides, for example, can be expressed heterologously or native to the cell, e.g., the adipocyte. Determining the ability of the test compound to modulate the activity of the polypeptide encoded by a gene involved in glucose transport (or biologically active portions thereof) can be accomplished by assaying for any one of the activities ascribed to such polypeptides as is standardly known in the art, for example, the activities of Akt1 or Akt2 as described herein (e.g., by assaying for GLUT4 trafficking). Determining the ability of the test compound to modulate the activity of a polypeptide encoded by a gene involved in glucose transport (or biologically active portions thereof) can also be accomplished by assaying for the activity of a substrate target molecule of that polypeptide. In one embodiment, determining the ability of the test compound to modulate the activity of a polypeptide encoded by a gene involved in glucose transport, or biologically active portion thereof, is accomplished by assaying for the ability to bind the substrate target molecule of the polypeptide or biologically active portions thereof. In a preferred embodiment, the cell overexpresses the polypeptide encoded by a gene involved in glucose transport, or biologically active portion thereof.

As used herein, the term “bioactive” fragment includes any portion (e.g., a segment of contiguous amino acids) of a polypeptide involved in glucose transport sufficient to exhibit or exert at least one glucose-transport associated activity including, for example, insulin-mediated glucose uptake by a cell. In various embodiments, the gene involved in glucose transport may be, for example, Akt1, Akt2, CISK or Myo1c.

According to the cell-based assays of the present invention, determining the ability of the test compound to modulate the activity of the polypeptide involved in glucose transport, or biologically active portion thereof, can be determined by assaying for a native activity, or an indirect activitiy codincident to the activity of an Akt, CISK or Myo1c polypeptide, as described herein. For example, the effect of the test compound on the ability of an Akt-expressing cell to uptake glucose in an insulin-dependent manner can be assayed in the presence of the test compound. It is also intended that in preferred embodiments, the cell-based assays of the present invention comprise a final step of identifying a test compound as a modulator of insulin response.

B. High Throughput Assays

High-throughput assays that employ targeted gene knockout in adipocytes are also useful for testing targets. For example, pools of different siRNAs targeted to nucleic acid sequences expressed in adipocytes are transfected into adipocytes using the methods described herein. The transfected cells are then tested for perturbation of one or more functions (e.g., glucose transport or GLUT4 translocation). Pools of siRNAs demonstrating such perturbations are further divided into smaller pools and the process is repeated until the sequence or sequences that are associated with the observed perturbation (effect) are identified. This method can also be useful for identifying effects that require the knockdown of more than one sequence since more than one type of siRNA may be taken up by a cell. Such techniques are useful, e.g., for investigations related to drug discovery for adipocyte-related diseases such as diabetes and obesity. For example, such methods can be used to create libraries of drug targets useful for studying functions and disorders associated with adipocytes such as glucose transport and diabetes.

VI. Test Compounds

The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc Natl Acad Sci USA 89:1865-1869) or on phage (Scott and Smith (1990) Science 249:386-390); (Devlin (1990) Science 249:404-406); (Cwirla et al. (1990) Proc. Natl. Acad. Sci. 87:6378-6382); (Felici (1991) J. Mol. Biol. 222:301-310); (Ladner supra.).

In a preferred embodiment, the library is a natural product library.

VII. Pharmaceutical Compositions

This invention further pertains to insulin response modulators identified by the above-described screening assays. Insulin response modulators identified by the above-described screening assays can be tested in an appropriate animal model. For example, an insulin response modulator identified as described herein can be used in an animal model to determine the efficacy, toxicity, or side effects of treatment with such a modulator. Alternatively, a modulator identified as described herein can be used in an animal model to determine the mechanism of action of such an agent. Furthermore, this invention pertains to uses of insulin response modulators identified by the above-described screening assays for therapeutic treatments as described infra.

Accordingly, the insulin response modulators of the present invention can be incorporated into pharmaceutical compositions suitable for administration. Such compositions typically comprise the nucleic acid molecule, protein, antibody, or modulatory compound and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

The pharmaceutical compositions can be included in a container, pack, or dispenser together with instructions for administration.

VIII. Methods of Treatment

The present invention also features methods of treatment or therapeutic methods. In one embodiment, the invention features a method of treating a subject (e.g., a human subject in need thereof) with a modulatory compound identified according to the present invention, such that a desired therapeutic effect is achieved. In another embodiment, the method involves administering to an isolated tissue or cell line from the subject a modulatory compound identified according to the methodology described herein, such that a desired therapeutic effect is achieved. In a preferred embodiment, the invention features a method of treating a subject having an insulin response disorder, for example, reduced insulin sensitivity or insulin resistance or diabetes (e.g., Type II diabetes). The present invention also provides for therapeutic methods of treating a subject having pre-diabetes or symptoms thereof, hyperglycemia and/or Type I diabetes. A preferred therapeutic effect is modulation of glucose uptake and/or transport. Desired therapeutic effects also include, but are not limited to curing or healing the subject, alleviating, relieving, altering or ameliorating a disease or disorder in the subject or at least one symptom of said disease or disorder in the subject, or otherwise improving or affecting the health of the subject. A preferred aspect of the invention pertains to methods of modulating genes identified by the methods provided herein as affecting glucose transport, or to known genes which affect glucose transport or insulin responsiveness, for therapeutic purposes.

The modulators identified by the methods disclosed herein may be used in a subject to modulate insulin responsiveness, regulate glucose transport, regulate gluconeogenesis, regulate glucose homeostasis, and to regulate blood glucose levels.

The effectiveness of treatment of a subject with an insulin response modulator can be accomplished by (i) detecting the level of insulin responsiveness or, alternatively, glucose tolerance in the subject prior to treating with an appropriate modulator; (ii) detecting the level of insulin responsiveness or, alternatively, glucose tolerance in the subject prior post treatment with the modulator; (iii) comparing the levels pre-administration and post administration; and (iv) altering the administration of the modulator to the subject accordingly. For example, increased administration of the modulator may be desirable if the subject continues to demonstrate insensitive insulin responsiveness.

Alternatively, the effectiveness of treatment of a subject with an insulin response modulator can be accomplished by (i) detecting the blood glucose or glucose tolerance in the subject prior to treating with an appropriate modulator; (ii) detecting the blood glucose level or, alternatively, glucose tolerance in the subject prior post treatment with the modulator; (iii) comparing the levels pre-administration and post administration; and (iv) altering the administration of the modulator to the subject accordingly. For example, increased or sustained administration of the modulator may be desirable if the subject fails to adequately clear blood glucose.

IX. Knockout and/or Knockdown Cells or Organisms

A further preferred use for the siRNA molecules of the present invention (or vectors or transgenes encoding same) is a functional analysis to be carried out in eukaryotic cells, or eukaryotic non-human organisms, preferably mammalian cells or organisms and most preferably human cells. By administering a suitable siRNA molecules which is sufficiently complementary to a target mRNA sequence to direct target-specific RNA interference, a specific knockout or knockdown phenotype can be obtained in a target cell, e.g. in cell culture, such as in adipocytes, or in a target organism.

Thus, a further subject matter of the invention is a eukaryotic cell, e.g., an adipocyte, or a eukaryotic non-human organism exhibiting a target gene-specific knockout or knockdown phenotype comprising a fully or at least partially deficient expression of at least one endogeneous target gene wherein said cell or organism is transfected (e.g., by electroporation) with at least one vector comprising DNA encoding a siRNA molecule capable of inhibiting the expression of the target gene. It should be noted that the present invention allows a target-specific knockout or knockdown of several different endogeneous genes due to the specificity of the siRNAi.

Gene-specific knockout or knockdown phenotypes of cells or non-human organisms, particularly of human cells or non-human mammals may be used in analytic to procedures, e.g. in the functional and/or phenotypical analysis of complex physiological processes such as analysis of gene expression profiles and/or proteomes. Preferably the analysis is carried out by high throughput methods using oligonucleotide based chips.

Using RNAi based knockout or knockdown technologies, the expression of an endogeneous target gene may be inhibited in a target cell or a target organism. The endogeneous gene may be complemented by an exogenous target nucleic acid coding for the target protein or a variant or mutated form of the target protein, e.g. a gene or a DNA, which may optionally be fused to a further nucleic acid sequence encoding a detectable peptide or polypeptide, e.g. an affinity tag, particularly a multiple affinity tag.

Variants or mutated forms of the target gene differ from the endogeneous target gene in that they encode a gene product which differs from the endogeneous gene product on the amino acid level by substitutions, insertions and/or deletions of single or multiple amino acids. The variants or mutated forms may have the same biological activity as the endogeneous target gene. On the other hand, the variant or mutated target gene may also have a biological activity, which differs from the biological activity of the endogeneous target gene, e.g. a partially deleted activity, a completely deleted activity, an enhanced activity etc. The complementation may be accomplished by compressing the polypeptide encoded by the endogeneous nucleic acid, e.g. a fusion protein comprising the target protein and the affinity tag and the double stranded RNA molecule for knocking out the endogeneous gene in the target cell. This compression may be accomplished by using a suitable expression vector expressing both the polypeptide encoded by the endogenous nucleic acid, e.g. the tag-modified target protein and the double stranded RNA molecule or alternatively by using a combination of expression vectors. Proteins and protein complexes which are synthesized de novo in the target cell will contain the exogenous gene product, e.g., the modified fusion protein. In order to avoid suppression of the exogenous gene product by the siRNAi molecule, the nucleotide sequence encoding the exogenous nucleic acid may be altered at the DNA level (with or without causing mutations on the amino acid level) in the part of the sequence which so is homologous to the siRNA molecule. Alternatively, the endogeneous target gene may be complemented by corresponding nucleotide sequences from other species, e.g. from mouse.

### Uses

The methods described herein are useful for identifying genes whose expression affects adipocyte development or metabolism. Furthermore, by using the methods which knock down specific genes, the effects of such knockdown on a cell can be characterized as useful in screening for drugs. For example, the desired effects of a drug that specifically affects expression or activity of a protein expressed in an adipocyte can be predicted by examining adipocytes in which the sequence encoding the protein has been knocked down. The phenotypes of such cells provide guidance for identifying the characteristics of a drug that is useful for inhibiting expression or activity of the targeted gene.

### EXAMPLES

### Example 1

### Materials and Methods

Materials

Human insulin was obtained from Eli Lilly Co. Goat polyclonal anti-Akt1 antibody (antigen human Akt1 peptide near C terminus, sc-7126), HRP-conjugated donkey anti-goat IgG, mouse monoclonal anti-lamin A/C (sc7293), and monoclonal anti-GSK3α/β were from Santa Cruz Biotech (Santa Cruz, Calif.). Rabbit polyclonal anti-Akt2 antibody (antigen peptide at C terminal of human Akt2) was provided by Dr. Morris J. Birnbaum (University of Pennsylvania; see Hill et al., 1999, Mol. Cell. Biol. 19:7771-7781). Rabbit polyclonal antibody against Acrp30 was from Affinity Bioreagents, Inc (Golden, Colo.) and antibody against nonmuscle myosin IIB was from Covance (Richmond, Calif.). Polyclonal antibodies against phospho-Akt threonine 308/309, phospho-GSK3 α/β (ser21/9), and phospho-Erk1/2 were from Cell Signaling Technology (Beverly, Mass.). Fluorescein isothiocyanate (FITC)-conjugated goat anti-mouse antibody was from Biosource International (Camarillo, Calif.).

Design and Synthesis of siRNA Duplexes

21-mer sense and antisense strands of RNA oligonucleotides were designed as described in Elbashir et al., 2001, Nature 411:94. The RNA oligonucleotides were synthesized with a nucleic acid synthesizer and protected with 2′-orthoester to prevent nuclease degradation followed by purification with anion exchange HPLC at Dharmacon Research, Inc. The sense and antisense strands of RNA oligonucleotides were then 2′-deprotected, annealed, and lyophilized to produce siRNA. The siRNA duplexes were desalted by washing with 75% ethanol twice, dried using a Speed-Vac, and the pellets were resuspended in nuclease-free water before transfection into cultured cells.

Cell Culture and Electroporation of 3T3L1 Adipocytes

3T3-L1 fibroblasts were grown in DMEM medium supplemented with 10% fetal bovine serum, 50 μg/ml streptomycin, and 50 units/ml penicillin. They were differentiated into adipocytes as described previously in Harrison et al. (1990, J. Biol. Chem. 265:20106). 3T3-L1 adipocytes were transfected with siRNA duplexes by electroporation. Briefly, at day 5 of differentiation the adipocytes were detached from culture dishes with 0.25% trypsin and 0.5 mg of collagenase per ml in phosphate-buffered saline (PBS), washed twice, and resuspended in PBS. About 5 million cells (half of the cells from one p150 culture dish) were then mixed with siRNA duplexes that were delivered to the cells by a pulse of electroporation with a Bio-Rad Gene Pulser II system (Bio-Rad Laboratories, Hercules, Calif.) at a setting of 0.18 kV and 960 μF capacitance. The electroporation was carried out using 450 μL of cells. After electroporation, cells were immediately mixed with fresh medium and incubated at 37° C. for 10 minutes before reseeding onto multiple-well plates designed for the deoxyglucose uptake assay, Western blotting, and immunofluorescence microscopic analysis. Analyses were generally carried out 24 or 48 hours after reseeding.

Immunofluorescence Microscopy

To visualize lamin A/C, cells were fixed with 4% formaldehyde and permeabilized with PBS containing 1% FBS and 0.5% Triton™ X-100. Cells were then incubated with primary mouse anti-rat lamin A/C antibody overnight at 4° C. After washing, the cells were incubated with FITC-labeled goat anti-mouse IgG for 30 minutes at room temperature. After washing, the coverslips were mounted in 90% glycerol containing 2.5% DABCO. Fluorescence microscopy was carried out with an IX70 inverted microscope (Olympus America, Inc, Melville, N.Y.) with CCD camera (Roper Scientific, Inc, Trenton, N.J.), and Metamorph image processing software (Universal Imaging Corp., Downington, Pa.).

Western Blotting

Following experimental treatments, the cells were solubilized in 25 mM Hepes, pH7.4, 1% NP40, 100 mM NaCl, 5 mM sodium fluoride, 1 mM EDTA, 1 mM sodium vanadate, 5 mM sodium pyrophosphate, 1 mM phenylmethylsulfonyl fluoride (PMSF), 10 μg/ml aprotinin, and 5 μg/ml leupeptin. To detect phosphorylation of Akt threonine308/309, GSK3 α/β serine21/9, and tyrosine phosphorylation of Erk1/2; 50 μg protein from 3T3-L1 adipocyte lysates were resolved with 8% SDS-PAGE and electrotransferred to nitrocellulose membranes. The membranes were then incubated with anti-phospho-specific antibodies (1:1000 dilution) overnight at 4° C. and then incubated in horseradish peroxidase (HRP)-linked anti-rabbit Ig antibodies (1:10,000 dilution) for 1 hour at room temperature. The membranes were washed with wash buffer (PBS pH 7.4, 0.1% Tween 20) for 1 hour at room temperature after incubation with each antibody. Finally, the level of serine phosphorylated Akt was detected using an ECL™ kit. Akt1 was detected with primary goat polyclonal antibody (1:750 dilution) and secondary HRP-linked donkey anti-goat antibody (1:10,000 dilution). Primary rabbit polyclonal antibodies against Akt2 (1:1000 dilution), nonmuscle myosin IIB (0.1 μg/ml) and Acrp30 (0.5 μg/ml) were used to detect their antigens using 25 μg protein from total cell lysates. To use the same nitrocellulose membrane to detect several proteins and phospho-proteins, the blots were incubated with gentle shaking in stripping buffer (62.5 mM tris-HCl, pH 6.7, 100 mM 2-mercapotoethanol and 2% SDS) for 30 to 45 minutes at 60° C. and washed for at least 1 hour with wash buffer before reblotting with the antibody designed for the next experiment.

2-Deoxyglucose Uptake Assay

Insulin-stimulated glucose transport in 3T3-L1 adipocytes was estimated by measuring 2-deoxyglucose uptake. Briefly, siRNA transfected cells were reseeded on 12-well plates, cultured for 40 hours, and then washed twice with DMEM medium before incubation with DMEM medium containing 0.5% bovine serum albumin (BSA) for 4 hours at 37° C. Cells were then washed twice with Krebs-Ringer Hepes (KRH) buffer (130 mM NaCl, 5 mM KCl, 1.3 mM CaCl₂, 1.3 mM MgSO4, 25 mM Hepes [pH 7.4]) and further starved for 1.5 hour in KRH buffer supplemented with 0.5% BSA and 2 mM sodium pyruvate. Cells were then stimulated with insulin for 30 minutes at 37° C. Glucose uptake was initiated by addition of [1,2-³H] 2-deoxy-D-glucose to a final assay concentration of 100 μM for 5 minutes at 37° C. Assays were terminated by four washes with ice-cold KRH buffer and the cells were solubilized with 0.4 ml of 1% Triton™ X-100, and the amount of tritium uptake was determined by scintillation counting. Nonspecific deoxyglucose uptake was measured in the presence of 20 μM cytochalasin B and was subtracted from each determination to obtain specific uptake.

siRNA-Induced Degradation of Myo1c

Either 20 nmol of scrambled or siRNA 6 alone, or 10 mmol each of siRNA 2 and siRNA 6 were electroporated into differentiated 3T3-L1 adipocytes. After electroporation the cells were reseeded in 24-well plates and allowed to rest for 24 hours. Glucose uptake was then measured. The fold increase was calculated after normalizing the basal uptake in scrambled siRNA-transfected cells to 1 and using that as a control. A paired t-test was used to analyze the percentage decrease in 2-deoxyglucose uptake in presence of siRNA 6 alone versus siRNA 6 and siRNA2 combined. A portion of these cells was analyzed for Myo1c, myosin 2b and EHD2 by western blotting.

### Example 2

### Introduction of siRNA into Adipocytes

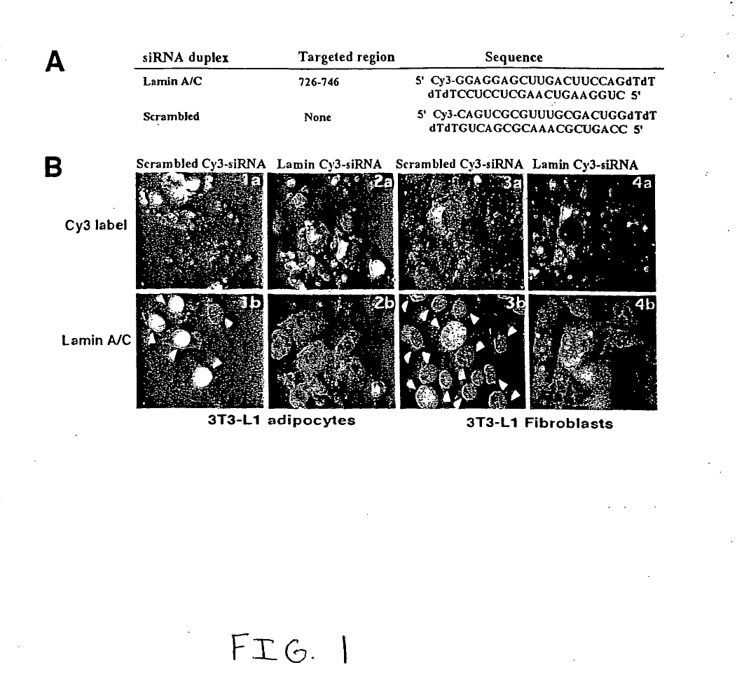

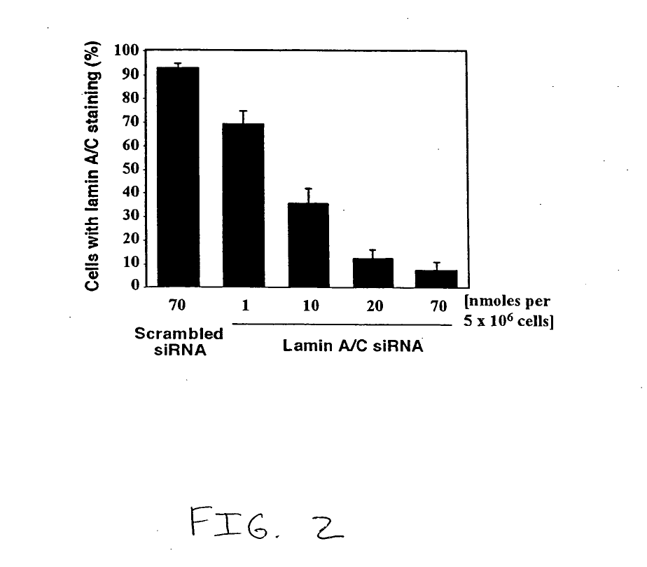

Initial experiments were performed that demonstrated that conditions developed for siRNA-mediated gene silencing in other cells types (Elbashir et al., 2001, Nature 411:94) worked well in 3T3-L1 fibroblasts (FIG. 1B, right panels), but failed work in 3T3-L1 adipocytes. Other means of transfection which are effective in other cell types, such as Oligofectamine™, showed considerable toxicity towards adipocytes.

An alternate methodology was developed to apply techniques using siRNA to adipocytes. The technique was developed using electroporation of 3T3-L1 adipocytes and Cy3-tagged siRNA duplexes targeted to mouse lamin A/C (FIG. 1A; sense and antisense strands, SEQ ID NOs: 1 and 2, respectively, targeting mouse lamin A/C of SEQ ID NO: 19) using the method described in Example 1. With this new technique, Cy3-siRNA was introduced with virtually 100% efficiency into the cultured adipocytes and by 48 hours nearly all cells showed loss of nuclear lamin A/C compared to cells transfected with a scrambled Cy3-tagged siRNA species (sense and antisense strands of SEQ ID NOs. 3 and 4, respectively) (FIG. 1B, left panels). Quantification of these results showed that adding 20 nmoles siRNA to a suspension of 5×10⁶ adipocytes resulted in the loss of lamin A/C in about 90% of the adipocytes with no detectable toxicity (FIG. 2). These findings provide the basis of a method for reliable and efficient gene silencing with low toxicity in insulin-sensitive cultured adipocytes.

Virtually all cells in the culture show decreased expression of the protein of interest in response to siRNA-directed gene silencing. This differs from the expression of cDNA-encoded proteins following transfection of plasmid vectors in adipocytes, where in most cases only 1-10% of cells express the heterologous protein. Thus siRNA-directed gene silencing using this method is valuable for assessing the role of a selected gene product in functions where single cell assays are difficult. One example is insulin-stimulated glucose transport. The new method of introducing nucleic acids such as siRNAs into adipocytes can also be used, e.g., in conjunction with single cell assays such as GLUT4 translocation assessed with the use of myc-tagged GLUT-GFP.

### Example 3

### Inhibition of Akt Expression

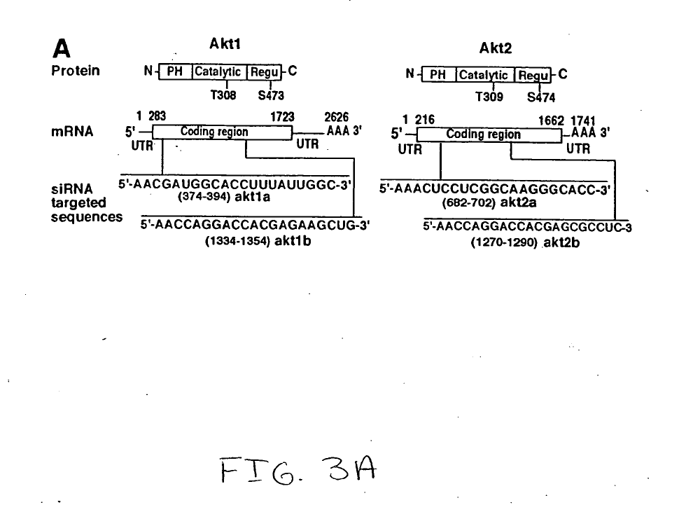

To further investigate the new method of introducing nucleic acids into adipocytes, two siRNA species directed against each of the Akt isoforms Akt1 and Akt2 were tested for their abilities to inhibit expression of these protein kinases in 3T3-L1 adipocytes (FIGS. 3A-B). Akt1 and Akt2 siRNA duplexes were designed according to mouse Akt1 and Akt2 mRNA sequences obtained from the National Center for Biotechnology Information database (accession nos. NM_{—}009652 and U22445, respectively) (SEQ ID NOs: 20 and 21, respectively).

The siRNAs targeted to Akt 1 were targeted to one of two target sequences in Akt1, “akt1a” and “akt1b”, as set forth in FIG. 2 (5′-AACGAUGGCACCUUUAUUGGC; SEQ ID NO: 5 and 5′-AACCAGGACCACGAGAAGCUG; SEQ ID NO: 6). The antisense strand of the siRNA targeting sequence “akt1a” had the sequence of 5′-GCCAAUAAAGGUGCCAUCGdTdT (SEQ ID NO: 9) and the antisense strand of the siRNA targeting sequence “akt1b” had the sequence of 5′-CAGCUUCUCGUGGUCCUGGdTdT (SEQ ID NO: 10). The siRNAs targeted to Akt2 were targeted to one of two target sequences in Akt2, “akt2a” and “akt2b”, as set forth in FIG. 2 (5′-AAACUCCUCGGCAAGGGCACC; SEQ ID NO: 7 and 5′-AACCAGGACCACGAGCGCCUC; SEQ ID NO: 8). The antisense strand of the siRNA targeting sequence “akt1a” had the sequence of 5′-GGUGCCCUUGCCGAGGAGUdTdT (SEQ ID NO: 11) and the antisense strand of the siRNA targeting sequence “akt1b” had the sequence of 5′-GAGGCGCUCGUGGUCCUGGdTdT (SEQ ID NO: 12).

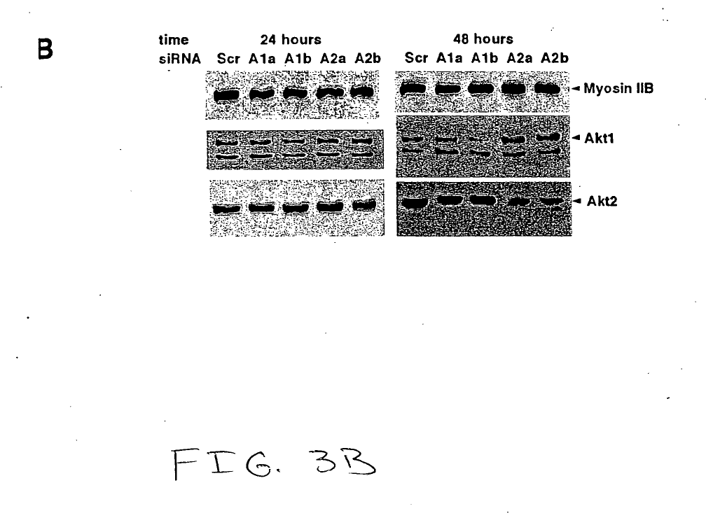

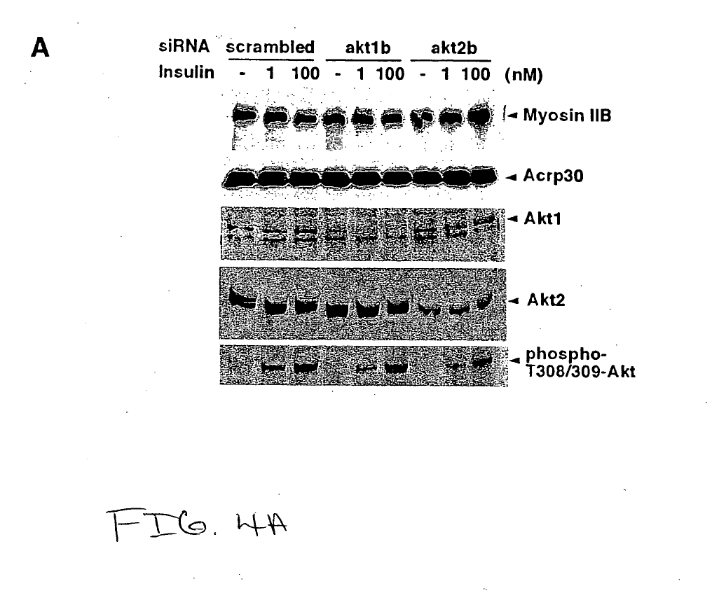

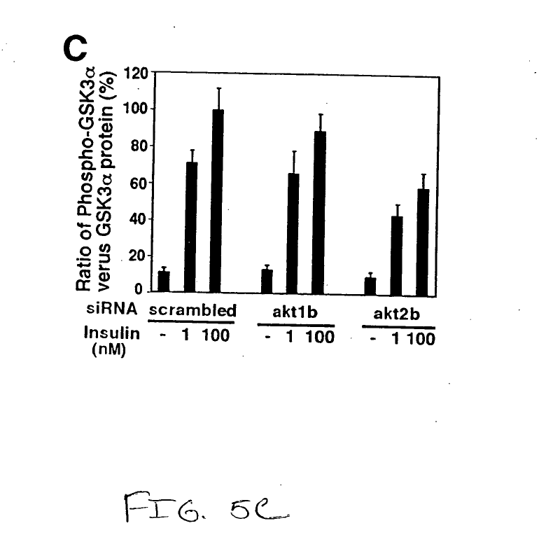

The siRNAs targeted to these sequences were introduced into the cells as described in Example 1. Each of the Akt 1-directed siRNA species inhibited expression of Akt1 protein at both 24 and 48 hours after transfection. One of these (akt1b) directed virtually total ablation of Akt1 expression by 48 hours, while Akt2 expression was unaffected (FIGS. 3B, 4A and B). Similarly, Akt2 expression could be selectively attenuated by about 70% after transfection of the siRNA species akt2b, while the akt2a siRNA was less effective (FIGS. 3B, 4A, and 4B). The akt1b and akt2b siRNA species that were most effective were targeted to similar regions of the Akt1 and Akt2 mRNA sequences; sequences that encode amino acids 351-357 and 352-358 in Akt1 and Akt2, respectively (FIG. 3A). The selectivity of akt1b versus akt2b siRNAs to silence their respective target mRNA species is apparent even though only 4 out of 21 nucleotides are different (FIG. 1A). This demonstrates the specificity of using these siRNAs for targeting these similar but distinct, sequences. Expression of several other unrelated proteins (e.g., myosin IIb shown in FIGS. 3, 4A, and 6A, and Acrp30 which is shown in FIGS. 4A and 6A) were unaffected by akt1b or akt2b siRNAs, further demonstrating the specificity of the method.

Akt1 and Akt2 are phosphorylated and activated by the protein kinase PDK1 at threonine 308 or 309 in the activation T-loop, and further activation occurs through phosphorylation at serine 473 (Akt1) or 474 (Akt2) (Alessi et al., 1997, Curr. Biol. 7:776; Williams et al., 2001, Curr. Biol. 10:439; Brazil et al., 2001, Trends Biochem. Sci. 26:657). The effects of selective loss of Akt1 versus Akt2 proteins on insulin-stimulated phosphorylation of total threonine 308/309 contained in both proteins were assessed by Western blotting with an antiphospho-Thr308 antibody. Total loss of Akt1 protein resulted in only a 10-20% reduction in total Thr308 phosphorylation of Akt protein kinases in cultured 3T3-L1 adipocytes, consistent with previous results showing Akt1 is much less abundant than Akt2 in adipocytes (Hill et al., 1999, Mol. Cell. Biol. 19:7771; Summers et al., 1999, J. Biol. Chem. 274:23858; Calera et al., 1998, J. Biol. Chem. 273:7201). In contrast, reduction of Akt2 expression by about 70% caused a marked 55-60% decrease in insulin-stimulated threonine phosphorylation of the Akt protein kinases (FIG. 4). Taken together, these data confirm the predominance of Akt2 over Akt1 in insulin-sensitive cultured adipocytes.

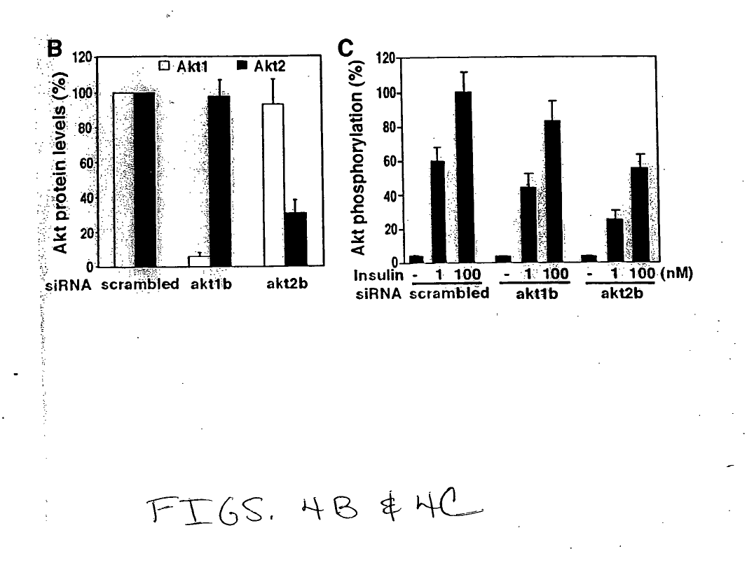

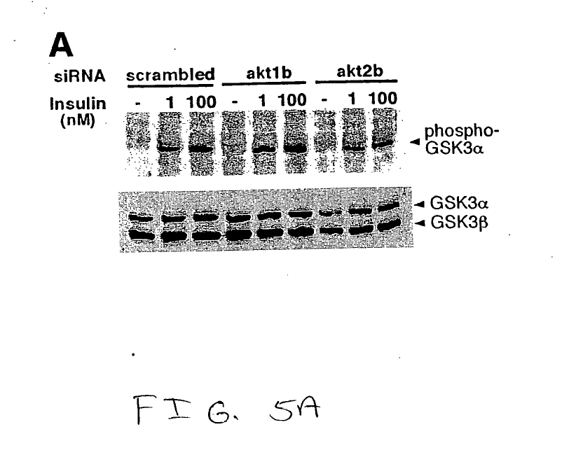

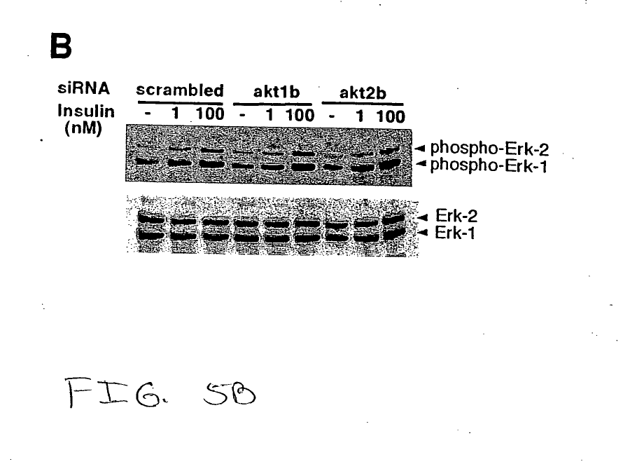

The consequences of the selective attenuation of Akt1 or Akt2 expression on a downstream target of insulin signaling-glycogen synthase kinase (GSK) 3 (Cross et al., 1995, Nature 378:785; Cohen et al., 2001, Nat. Rev. Mol. Cell. Biol. 2:769) were assessed. GSK3α is thought to be phosphorylated by Akt protein kinases in response to the hormone in a dose-dependent manner (FIGS. 5A and 5B). In three independent experiments, loss of 95% or more of Akt1 directed by the akt1b siRNA caused no significant attenuation of insulin-mediated GSK3α phosphorylation (FIGS. 5A and 5B), although a 10-20% effect may go undetected in these studies. In contrast, attenuation of Akt2 expression by about 70% caused about a 40% inhibition of insulin-mediated GSK3α phosphorylation (FIGS. 5A, 5B, 6A, and 6C). In control studies, no diminution of insulin signaling to the MAP kinases Erk-1 and Erk-2 was observed when either Akt1 or Akt2 were depleted (FIGS. 5C and 5D), confirming the specificity of the effect of silencing the Akt protein kinases by this method. These data indicate that insulin action on GSK3α in cultured adipocytes specifically requires Akt2. Thus, inhibition of expression or activity of Akt2 can be used to inhibit insulin-mediated GSK3α phosphorylation.

Applying this same approach to hexose transport regulation, it was found that ablation of Akt1 expression (FIG. 6A) leads to a small but significant 20-30% decrease in insulin-stimulated 2-deoxyglucose uptake in 3T3-L1 adipocytes (FIG. 6B). Akt2 protein depletion to about 70% of normal levels dampened the insulin response by 50-58%. These data indicate that both Akt1 and Akt2 contribute to insulin responsiveness of hexose transport in cultured adipocytes roughly in proportion to their contributions to total activated Akt in these cells (FIG. 4). The effects were tested of depleting both Akt1 and Akt2 in 3T3-L1 adipocytes using the combination of akt1b and akt2b siRNA species (FIGS. 6A and 6C). This combined treatment virtually completely ablated Akt1 expression and reduced Akt2 expression by over 65% while insulin-stimulated phosphorylation of total Akt detected by anti-phospho threonine 308/309 antibody decreased by 81% (FIG. 6A). Importantly, insulin-stimulated deoxyglucose uptake was inhibited by nearly 80% under these conditions, compared to about 58% when only Akt2 was depleted (FIG. 6C). Under these conditions, GLUT4 expression was unchanged.

These data demonstrate that while Akt2 is the major protein kinase in this response, Akt1 also can play an important role. Thus, under conditions where insulin-stimulated glucose uptake is significantly compromised by partial depletion of Akt2, Akt1 is required for half of the remaining insulin signal (FIG. 6C). GSK3α phosphorylation in response to insulin was also inhibited to a greater extent when both protein kinases were depleted versus when only Akt2 was reduced (FIGS. 6A and 6C).

These data also suggest that increasing Akt1 or Akt expression or activity is useful for stimulating insulin-stimulated glucose uptake, e.g., when Akt1 or Akt2 expression or activity is deficient. This also demonstrates the usefulness of the method of introducing an siRNA into an adipocyte to identify targets for treatment of adipocyte-related disorders such as diabetes.

The findings presented here show an absolute requirement of Akt protein kinases for normal insulin signaling to glucose transport and GSK3α and imply direct proportionality of total available Akt1 plus Akt2 to the degree of insulin responsiveness (FIG. 6). This conclusion is in keeping with the similar insulin dose response relationships observed for activation of total Akt and glucose transport (compare FIGS. 4C and 6B). Progressive loss of Akt1, Akt2, or both leads to a correspondingly progressive loss in glucose transport stimulation (FIG. 6). These considerations show that Akt1 can, in part, replace Akt2 to sustain glucose transport responsiveness. Thus, methods that increase the expression or activity or Akt1 are useful for increasing glucose transport responsiveness.

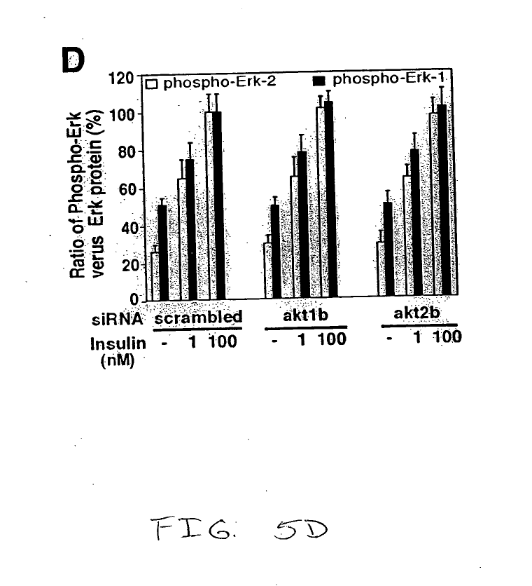

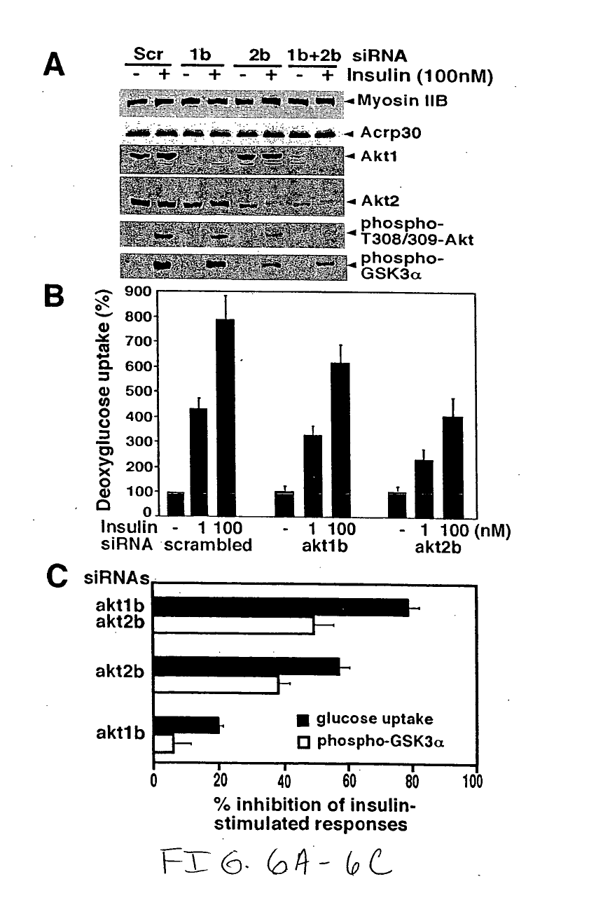

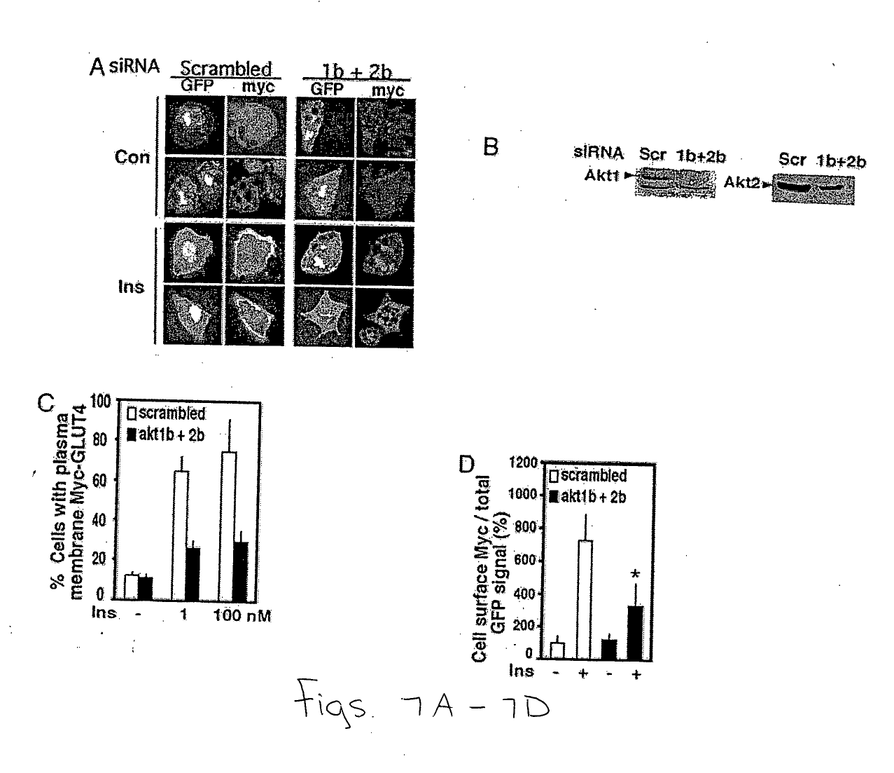

The effect of combined depletion of both Akt1 and Akt2 by siRNA on insulin-mediated GLUT4 translocation was next examined in 3T3-L1 adipocytes. Cotransfection of myc-GLUT4-EGFP plasmid DNA with the mixture of akt1b and akt2b siRNAs was performed, and 48 hours later reductions of ˜90% and 65% of Akt1 and Akt2 protein levels, respectively, were observed (FIG. 7B). This combined knockdown of Akt1 and Akt2 proteins resulted in the loss of insulin-stimulated cell surface Myc signal (detected by anti-Myc Ab) in ˜70% of adipocytes transfected with the Myc-GLUT4-GFP construct (FIGS. 7A and C). Quantifying the ratio of cell surface Myc rim signal over the total Myc-GLUT4-GFP signal in positive transfected cells revealed that loss of both Akt1 and Akt2 resulted in a 60% decrease in insulin-stimulated Myc-GLUT4-GFP on the cell surface (FIG. 7D). The attenuation of GLUT4 responsiveness is observed at both maximal and submaximal concentrations of insulin (FIG. 7C), whereas no significant effect of Akt depletion on GLUT4-translocation in the absence of insulin is detected.

In summary, these data demonstrate the absolute requirement of Akt for insulin action on glucose transport. They also demonstrate the effectiveness of the methods for introducing nucleic acids, e.g., siRNA, into adipocytes.

### Example 4

### Silencing of Insulin Signaling Genes

Experiments were performed to test whether genes of particular relevance to insulin signaling can be silenced with siRNA in cultured adipocytes.

Western blot analysis of adipocyte lysates was performed following siRNA-directed gene silencing of the Akt-related protein kinase CISK. CISK contains a PX domain rather than a PH domain at the N terminus, but exhibits about 50% sequence identity with Akt in the protein kinase domain. CISK is activated by IGF-1 and EGF in COS cells, and activation of CISK in 3T3-L1 adipocytes can stimulate GLUT4 translocation. Therefore, the ability to reduce CISK expression by siRNA directed against sequences in its open reading frame was tested. In the case of such genes of interest several distinct siRNA species are generally prepared for testing. Candidate siRNAs directed against CISK were designed using methods known in the art and were tested using the method described herein. The experiments demonstrated that two such siRNA species targeting CISK were effective for silencing CISK expression. siRNAs targeting CISK were synthesized using standard techniques (Dharmacon, Inc.) Both siRNA CISK species that were tested were effective at selectively reducing CISK expression 48 hours after the transfection. At 24 hours, there was also a significant effect, although not as complete as at 48 hours. Transfection of the cultured adipocytes with the siRNA against lamin did not cause a reduction in CISK expression compared to cells that had not been transfected.

As another control, Western blotting of a nonrelevant protein, EHD2, which is also highly expressed in adipocytes, was performed. Expression of EHD2 showed little or no effect of the CISK-directed siRNA transfection.

Similarly, several siRNA species against the unconventional myosin Myo1c were tested. The siRNAs (Dharmacon) were designed to target the following Myo1c mRNA sequences: scrambled, 5′-CAGUCGCGUUUGCGACUGG (SEQ ID NO: 13); siRNA 2,5′-AAGGCGUUGUACAGCCGGACAUU (SEQ ID NO: 14); siRNA 6,5′-AAGCUUCCAGACAGGGAUCCAUG (SEQ ID NO: 15). The corresponding Myo1c siRNAs comprised an antisense strand having the following sequences: scrambled, 5′-CCAGUCGCAAACGCGACUGdTdT (SEQ ID NO: 16); siRNA 2,5′-UGUCCGGCUGUACAACGCCdTdT (SEQ ID NO: 17); and siRNA 6,5′-UGGAUCCCUGUCUGGAAGCdTdT (SEQ ID NO: 18).

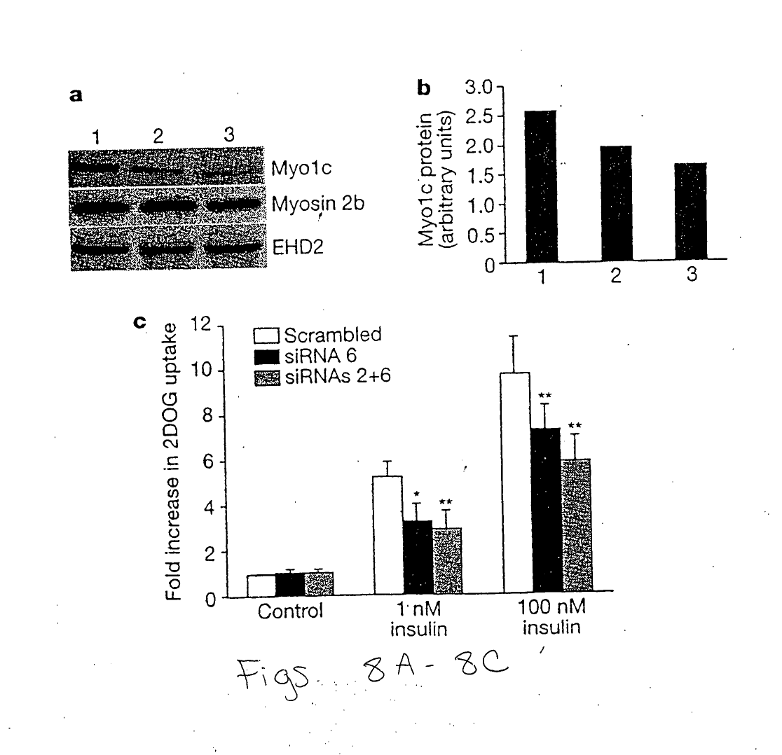

Twenty four hours after transfection, the amounts of Myo1c protein decreased by about 40% in cells transfected with a combination of two siRNA species and about 25% in cells transfected with one siRNA species (FIGS. 8A, B). Expression of two other control proteins, myosin 2b and EHD2, was unaffected (FIG. 8A). Insulin-stimulated 2-deoxyglucose uptake in adipocytes transfected with either siRNA 6 or both siRNA 6 and siRNA2 was significantly inhibited as compared with cells transfected with scrambled siRNA at concentrations of both 1 nM and 100 nM insulin. These results indicated that siRNA-mediated attenuation of Myo1c expression causes a corresponding decrease in GLUT4 responsiveness to insulin.

These data provide additional evidence that siRNA-directed gene silencing can be used in adipocytes for, e.g., gene discovery to evaluate the functions of proteins in the insulin signaling pathway and confirmation of drug targets.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of introducing a nucleic acid into an adipocyte, the method comprising:
(a) contacting an adipocyte having a cell membrane with a nucleic acid molecule, thereby forming a mixture; and
(b) electroporating the mixture under conditions such that the cell membrane becomes permeablized;
such that the nucleic acid is introduced into the adipocyte.

2. The method of claim 1, wherein the nucleic acid molecule is an siRNA.

3. The method of claim 1, wherein the electroporation is carried out at between about 0.05 kV and about 0.5 kV, and at between about 750 μF and about 1150 μF capacitance.

4. The method of claim 1, wherein the electroporation is carried out at between about 0.1 kV and about 0.25 kV, and at between about 850 μF and about 1050 μF capacitance.

5. The method of claim 1, wherein the electroporation is carried out at 0.18 kV and 960 μF capacitance.

6. The method of claim 1, wherein the electroporation is carried out at room temperature.

7. The method of claim 2, wherein the siRNA is targeted to an Akt1 or Akt2 nucleic acid sequence.

8. The method of claim 2, wherein the siRNA is targeted to a CISK nucleic acid sequence.

9. The method of claim 2, wherein the siRNA is targeted to a Myo1c nucleic acid sequence.

10. A method of inhibiting Akt1 expression or activity in a cell, the method comprising
(a) introducing into a cell expressing Akt1 an siRNA that targets an Akt1 sequence of SEQ ID NO:5 or SEQ ID NO: 6;
(b) culturing the cell under conditions such that the siRNA mediates RNAi;
thereby inhibiting Akt1 expression or activity in the cell.

11. The method of claim 10, wherein the cell is an adipocyte.

12. A method of inhibiting Akt1 expression or activity in an adipocyte, the method comprising:
(a) introducing into an adipocyte expressing Akt1 an siRNA that targets an Akt1 sequence of SEQ ID NO:5 or SEQ ID NO: 6, wherein the siRNA is introduced according to the method of claim 1;
(b) culturing the adipocyte under conditions such that the siRNA mediates RNAi;
thereby inhibiting Akt1 expression or activity in the cell.

13. A method of inhibiting Akt2 expression or activity into a cell, the method comprising
(a) introducing into a cell expressing Akt2 an siRNA that targets an Akt2 sequence of SEQ ID NO:7 or SEQ ID NO: 8;
(b) culturing the cell under conditions such that the siRNA mediates RNAi;
thereby inhibiting Akt2 expression or activity in the cell.

14. The method of claim 13, wherein the cell is an adipocyte.

15. A method of inhibiting Akt2 expression or activity in an adipocyte, the method comprising:
(a) introducing into an adipocyte expressing Akt2 an siRNA that targets an Akt2 sequence of SEQ ID NO:7 or SEQ ID NO: 8, wherein the siRNA is introduced according to the method of claim 1;
(b) culturing the adipocyte under conditions such that the siRNA mediates RNAi;
thereby inhibiting Akt2 expression or activity in the cell.

16. A method of inhibiting Myo1c expression or activity into a cell, the method comprising
(a) introducing into a cell expressing Myo1c an siRNA that targets an Myo1c sequence of SEQ ID NO:14 or SEQ ID NO: 15;
(b) culturing the cell under conditions such that the siRNA mediates RNAi;
thereby inhibiting Myo1c expression or activity in the cell.

17. The method of claim 13, wherein the cell is an adipocyte.

18. A method of inhibiting Myo1c expression or activity in an adipocyte, the method comprising:
(a) introducing into an adipocyte expressing Myo1c an siRNA that targets an Myo1c sequence of SEQ ID NO:14 or SEQ ID NO: 15, wherein the siRNA is introduced according to the method of claim 1;
(b) culturing the adipocyte under conditions such that the siRNA mediates RNAi;
thereby inhibiting Myo1c expression or activity in the cell.

19. A method of inhibiting insulin-mediated GSK3α phosphorylation, the method comprising contacting an insulin-sensitive cell with an agent that inhibits Akt2 expression or activity, such that insulin-mediated GSK3α phosphorylation is inhibited.

20. The method of claim 19, wherein the agent is an siRNA targeted to Akt2.

21. The method of claim 20, wherein the siRNA targets an Akt2 sequence of SEQ ID NO: 7 or SEQ ID NO: 8.

22. A method of inhibiting hexose transport, the method comprising contacting a cell that can conduct hexose transport with an agent that specifically inhibits expression or activity of Akt1, Akt2, or both, such that hexose transport is inhibited.

23. The method of claim 22, wherein the agent specifically inhibits expression or activity of Akt1 or Akt2 and hexose transport is partially inhibited.

24. The method of claim 22, wherein the agent is a siRNA specifically targeted to Akt1, Akt2, or both.

25. The method of claim 24, wherein the siRNA targeting Akt2 targets a sequence of SEQ ID NO: 7 or SEQ ID NO: 8.

26. The method of claim 24, wherein the siRNA targeting Akt1 targets a sequence of SEQ ID NO: 5 or SEQ ID NO: 6.

27. A method of identifying a gene that affects glucose transport, the method comprising:
(a) introducing into an adipocyte an siRNA targeted against the gene using the method of claim 1;
(b) culturing the cell under conditions suitable for expression of the targeted gene;
(c) assaying glucose transport in the cell, wherein a reduction in glucose transport indicates that the targeted gene affects glucose transport;
thereby identifying a gene that affects glucose transport.

28. A method for identifying an insulin response modulator, comprising:
(a) contacting a cell expressing a protein that affects glucose transport with a test compound, wherein the protein is encoded by a gene identified according to the method of claim 27, and
(b) determining the ability of the test compound to modulate an activity of the protein, such that the insulin response modulator is identified.

29. A method for identifying an insulin response modulator, comprising:
(a) contacting a cell expressing a protein that affects glucose transport with a test compound, wherein the protein is encoded by a gene identified according to the method of claim 27, and
(b) determining the ability of the test compound to modulate expression of the protein, such that the insulin response modulator is identified.

30. The method of claim 28 or 29, wherein the modulator is a positive modulator.

31. The method of claim 28 or 29, wherein the modulator is a negative modulator.

32. A modulator identified by the method of claim 28 or 29.

33. A pharmaceutical composition comprising the modulator of claim 32.

34. A method of regulating glucose homeostasis in a subject comprising administering to the subject an insulin response modulator of claim 32.

35. A method of regulating blood glucose levels in a subject comprising administering to the subject an insulin response modulator of claim 32.

36. A method of treating an insulin response disease or disorder comprising administering the pharmaceutical composition of claim 33.

37. The method of claim 36, wherein the disease or disorder is selected from the group consisting of Type II diabetes, insulin resistance and obesity.


SEQUENCE LISTING (141 sequences)

SEQ ID NO: 1 - RNA, 21 nt, Artificial Sequence
  siRNA
ggaggagcuugacuuccagnn

SEQ ID NO: 2 - RNA, 21 nt, Artificial Sequence
  siRNA
cuggaagucaagcuccuccnn

SEQ ID NO: 3 - RNA, 21 nt, Artificial Sequence
  siRNA
cagucgcguuugcgacuggnn

SEQ ID NO: 4 - RNA, 21 nt, Artificial Sequence
  siRNA
ccagucgcaaacgcgacugnn

SEQ ID NO: 5 - RNA, 21 nt, Mus musculus
aacgauggcaccuuuauuggc

SEQ ID NO: 6 - RNA, 21 nt, Mus musculus
aaccaggaccacgagaagcug

SEQ ID NO: 7 - RNA, 21 nt, Mus musculus
aaacuccucggcaagggcacc

SEQ ID NO: 8 - RNA, 21 nt, Mus musculus
aaccaggaccacgagcgccuc

SEQ ID NO: 9 - RNA, 21 nt, Artificial Sequence
  siRNA
gccaauaaaggugccaucgnn

SEQ ID NO: 10 - RNA, 21 nt, Artificial Sequence
  siRNA
cagcuucucgugguccuggnn

SEQ ID NO: 11 - RNA, 21 nt, Artificial Sequence
  siRNA
ggugcccuugccgaggagunn

SEQ ID NO: 12 - RNA, 21 nt, Artificial Sequence
  siRNA
gaggcgcucgugguccuggnn

SEQ ID NO: 13 - RNA, 19 nt, Mus musculus
cagucgcguuugcgacugg

SEQ ID NO: 14 - RNA, 23 nt, Mus musculus
aaggcguuguacagccggacauu

SEQ ID NO: 15 - RNA, 23 nt, Mus musculus
aagcuuccagacagggauccaug

SEQ ID NO: 16 - RNA, 21 nt, Artificial Sequence
  siRNA
ccagucgcaaacgcgacugnn

SEQ ID NO: 17 - RNA, 21 nt, Artificial Sequence
  siRNA
uguccggcuguacaacgccnn

SEQ ID NO: 18 - RNA, 21 nt, Artificial Sequence
  siRNA
uggaucccugucuggaagcnn

SEQ ID NO: 19 - DNA, 2354 bp, Mus musculus
ccacgcctgccaggagcgagcttcgccggctcgctgtccccctgagcagcctctgtccttctgtccaagtcccgcgcccttctcgggacccctgcccagcgggcagcactgtcaccctgccggccatggagaccccgtcacagcggcgcgccacccgcagtggggcgcaggccagctctaccccactgtcgcccactcggatcacccggctgcaggagaaggaggacctgcaggagctcaatgaccgcctggccgtgtacatcgatcgcgtgcgttccctggagaccgagaacgcggggctgcgccttcgcatcactgagtctgaagaggtggtcagccgagaggtgtccggcatcaaggcggcctacgaggccgagctgggggatgcccgcaagacccttgattctgtggccaaggagcgcgcccgcctccagctagagctgagcaaagtgcgtgaggagttcaaggagctgaaggctcgcaacaccaagaaggagggggacttgttggctgcgcaggcccggctcaaggacctcgaggctcttctcaactccaaggaagctgccctgagcactgctctcagtgagaagcgcacattggagggcgagctccatgacctgcgggggcaggtagccaagcttgaggcggccctgggagaggctaagaagcagcttcaggatgagatgctgaggcgagtggatgctgagaacaggctacagacgctgaaggaggagcttgacttccagaagaacatttacagcgaggaactgcgtgagaccaagcgccggcatgagacgcggcttgtggagatcgataacgggaagcagcgagagtttgagagccggctggcagatgccctgcaggagctgcgggctcagcatgaggaccaggtggaacagtataagaaggagctagaaaagacatactccgccaagctggataatgccaggcagtctgctgagaggaacagcaacctcgtgggggctgcccatgaggaactgcagcagtctcgaatccgcattgacagcctctcggcccagctcagccagctccaaaagcagttggcagccaaggaggcaaagctgcgtgacctggaggactcgctggcccgtgagcgcgataccagccggcgcctgctggctgagaaagagcgagagatggcggagatgcgggcgaggatgcagcagcagctggacgagtaccaggagctgctggacatcaagctggccctggacatggagatccatgcctatcgaaagctgctggagggcgaggaggagaggctgcgcctgtcccccagccctacctcgcagcgcagccgtggccgcgcctcctcccactcatcccagtctcagggtggaggcagcgtcaccaaaaagcgcaagctggagtcttccgagagccggagcagcttctcgcagcatgctcgcactagcgggcgtgtggcggtagaggaagtcgatgaagagggaaagttcgtgcggctgcgcaacaagtccaacgaggaccagtccatgggcaactggcagatcaggcgtcagaatggtgacgatcctttgatgacctatcgcttcccaccgaagttcaccctaaaggctgggcaggtggtgacgatctgggcttcaggagctggggccacccatagcccccctactgacttggtgtggaaggcgcagaacacctggggctgtgggagcagccttcgcaccgctctcatcaactccactggagaagaagtggccatgcgcaagctggtgcgctcactgaccatggttgaggacaatgaggatgacgacgaggatggagaagagctcctccatcaccaccgtggttcccactgcagcggctcgggggaccccgctgagtacaacctgcgctcacgcaccgtgctgtgcgggacgtgtgggcagcctgctgacaaggctgccggtggagcgggagcccaggtgggcggatccatctcctctggctcttctgcctccagtgtcacagtcactcgaagcttccgcagtgtggggggcagtgggggtggcagcttcggggacaacctagtcacccgctcctacctcctgggcaactccagtccccggagccagagctcccagaactgcagcatcatgtaatctgggacctgccaggcagggctgggggcagaggccacctgctcccccctcaccacatgccacctcctgtctgctccttaggagagcaggcctgaagccaaagaaaaatttatcccctgcctttggttttttttttttttcttctattttttttttctttttctaagagaagttattttctacagtggttttatactgaaggaaaaactcaagcaaaaaaaaaaaaaaaaa

SEQ ID NO: 20 - DNA, 2626 bp, Mus musculus
ccgggaccagcggacggaccgagcagcgtcctgcggccggcaccgcggcggcccagatccggccagcagcgcgcgcccggacgccgctgccttcagccggccccgcccagcgcccgcccgcgggatgcggagcggcgggcgcccgaggccgcggcccggctaggcccagtcgcccgcacgcggcggcccgacgctgcggccaggccggctgggctcagcctaccgagaagagactctgatcatcatccctgggttacccctgtctctgggggccacggataccatgaacgacgtagccattgtgaaggagggctggctgcacaaacgaggggaatatattaaaacctggcggccacgctacttcctcctcaagaacgatggcacctttattggctacaaggaacggcctcaggatgtggatcagcgagagtccccactcaacaacttctcagtggcacaatgccagctgatgaagacagagcggccaaggcccaacacctttatcatccgctgcctgcagtggaccacagtcattgagcgcaccttccatgtggaaacgcctgaggagcgggaagaatgggccaccgccattcagactgtggccgatggactcaagaggcaggaagaagagacgatggacttccgatcaggctcacccagtgacaactcaggggctgaagagatggaggtgtccctggccaagcccaagcaccgtgtgaccatgaacgagtttgagtacctgaaactactgggcaagggcacctttgggaaagtgattctggtgaaagagaaggccacaggccgctactatgccatgaagatcctcaagaaggaggtcatcgtcgccaaggatgaggttgcccacacgcttactgagaaccgtgtcctgcagaactctaggcatcccttccttacggccctcaagtactcattccagacccacgaccgcctctgctttgtcatggagtatgccaacgggggcgagctcttcttccacctgtctcgagagcgcgtgttctccgaggaccgggcccgcttctatggtgcggagattgtgtctgccctggactacttgcactccgagaagaacgtggtgtaccgggacctgaagctggagaacctcatgctggacaaggacgggcacatcaagataacggacttcgggctgtgcaaggaggggatcaaggatggtgccactatgaagacattctgcggaacgccggagtacctggcccctgaggtgctggaggacaacgactacggccgtgcagtggactggtgggggctgggcgtggtcatgtatgagatgatgtgtggccgcctgcccttctacaaccaggaccacgagaagctgttcgagctgatcctcatggaggagatccgcttcccgcgcacactcggccctgaggccaagtccctgctctccgggctgctcaagaaggaccctacacagaggctcggtgggggctctgaggatgccaaggagatcatgcagcaccggttctttgccaacatcgtgtggcaggatgtgtatgagaagaagctgagcccacctttcaagccccaggtcacctctgagactgacaccaggtatttcgatgaggagttcacagctcagatgatcaccatcacgccgcctgatcaagatgacagcatggagtgtgtggacagtgagcggaggccgcacttcccccagttctcctactcagccagtggcacagcctgaggcctggggcagcggctggcagctccacgctcctctgcattgccgagtccagaagccccgcatggatcatctgaacctgatgttttgtttctcggatgcgctggggaggaaccttgccagcctccaggaccaggggaggatgtttctactgtgggcagcagcctacctcccagccaggtcaggaggaaaactatcctggggtttttcttaatttatttcatccagtttgagaccacacatgtggcctcagtgcccagaacaattagattcatgtagaaaactattaaggactgacgcgaccatgtgcaatgtgggctcatgggtctgggtgggtcccgtcactgcccccattggcctgtccaccctggccgccacctgtctctagggtccagggccaaagtccagcaagaaggcaccagaagcacctccctgtggtatgctaactggccctctccctctgggcggggagaggtcacagctgcttcagccctagggctggatgggatggccagggctcaagtgaggttgacagaggaacaagaatccagtttgttgctgtgtcccatgctgttcagagacatttaggggattttaatcttggtgacaggagagcccctgccctcccgctcctgcgtggtggctcttagcgggtaccctgggagcgcctgcctcacgtgagccctctcctagcacttgtccttttagatgctttccctctcccgctgtccgtcaccctggcctgtcccctcccgccagacgctggccattgctgcaccatgtcgttttttacaacattcagcttcagcatttttactattataataagaaactgtccctccaaattcaataaaaattgcttttcaagcttgaaaaaaaaaaaaaaaaaaaaaaaaaaaaaaa

SEQ ID NO: 21 - DNA, 1741 bp, Mus musculus
cggctcgcgccgccgccagcactgccgccgttgctgccgccagttcataaataaggagcgggaacgagctcagcgtggcgatgggcgggggtagagcccggccggagaggctgggcggccgccggtgacagacgatactgtatccgaggagcctcctgcatgtcctgctgccctgagctcactcaagctaggtgacagcgtgtgaatgctgccaccatgaatgaggtatctgtcatcaaagaaggctggctccacaaacgtggtgaatacatcaagacctggaggccacggtacttccttctgaagagtgatggatctttcattgggtataaggagaggcccgaggcccctgaccagaccttaccccccctgaacaatttctctgtagcagaatgccagctgatgaagactgagaggccacgacccaacacctttgtcatacgctgcctgcagtggaccacagtcatcgagaggaccttccatgtagactctccagatgagagggaagagtggatgcgggctatccagatggtcgccaacagtctgaagcagcggggcccaggtgaggacgccatggattacaagtgtggctcccccagtgactcttccacatctgagatgatggaggtagctgtcaacaaggcacgggccaaagtgaccatgaatgacttcgattatctcaaactcctcggcaagggcaccttcggcaaggtcattctggttcgagagaaggccactggccgctattatgccatgaagatcctgcgcaaggaggtcatcattgcaaaggatgaagtcgcccacacagtcacagagagccgggttctgcagaataccaggcaccccttccttacagccctcaagtatgccttccagacccatgaccgcctatgctttgtgatggagtatgccaacgggggtgagctgtttttccacctctctcgggagcgagtcttcacggaggatcgggcgcgcttttatggagcagagattgtgtcagctctggagtatttgcactcgagagatgtggtgtaccgtgacatcaagctggaaaaccttatgttggacaaagatggccacatcaagatcactgactttggcttgtgcaaagagggcatcagtgatggagccaccatgaaaaccttctgtggtaccccggagtacttggcgcctgaggtgctagaggacaatgactatgggcgagcagtggactggtgggggctgggtgtggtcatgtatgagatgatgtgtggccgcctgccattctacaaccaggaccacgagcgcctctttgagctcattcttatggaggagatccgcttcccgcgcacactcgggccagaggccaagtccctgctggctggactgctgaagaaggacccaaagcagaggctcggcggaggtcccagtgatgcgaaggaggtcatggagcatagattcttcctcagcatcaactggcaggacgtggtacagaaaaagctcctgccacccttcaaacctcaggtcacttcagaagtggacacaaggtactttgatgacgagttcaccgcccagtccatcacaatcacacccccagaccgatatgacagcctggacccgctggaactggaccagcggacgcacttcccccagttctcctactcagccagcatccgagagtgagcagccctctgccaccacaggacacaagcatggccgtcatccactgcctgggtggctttttaaaaaaaaaaaaaaaaag

SEQ ID NO: 22 - DNA, 2610 bp, Homo sapiens
atcctgggacagggcacagggccatctgtcaccaggggcttagggaaggccgagccagcctgggtcaaagaagtcaaaggggctgcctggaggaggcagcctgtcagctggtgcatcagaggctgtggccaggccagctgggctcggggagcgccagcctgagaggagcgcgtgagcgtcgcgggagcctcgggcaccatgagcgacgtggctattgtgaaggagggttggctgcacaaacgaggggagtacatcaagacctggcggccacgctacttcctcctcaagaatgatggcaccttcattggctacaaggagcggccgcaggatgtggaccaacgtgaggctcccctcaacaacttctctgtggcgcagtgccagctgatgaagacggagcggccccggcccaacaccttcatcatccgctgcctgcagtggaccactgtcatcgaacgcaccttccatgtggagactcctgaggagcgggaggagtggacaaccgccatccagactgtggctgacggcctcaagaagcaggaggaggaggagatggacttccggtcgggctcacccagtgacaactcaggggctgaagagatggaggtgtccctggccaagcccaagcaccgcgtgaccatgaacgagtttgagtacctgaagctgctgggcaagggcactttcggcaaggtgatcctggtgaaggagaaggccacaggccgctactacgccatgaagatcctcaagaaggaagtcatcgtggccaaggacgaggtggcccacacactcaccgagaaccgcgtcctgcagaactccaggcaccccttcctcacagccctgaagtactctttccagacccacgaccgcctctgctttgtcatggagtacgccaacgggggcgagctgttcttccacctgtcccgggaacgtgtgttctccgaggaccgggcccgcttctatggcgctgagattgtgtcagccctggactacctgcactcggagaagaacgtggtgtaccgggacctcaagctggagaacctcatgctggacaaggacgggcacattaagatcacagacttcgggctgtgcaaggaggggatcaaggacggtgccaccatgaagaccttttgcggcacacctgagtacctggcccccgaggtgctggaggacaatgactacggccgtgcagtggactggtgggggctgggcgtggtcatgtacgagatgatgtgcggtcgcctgcccttctacaaccaggaccatgagaagctttttgagctcatcctcatggaggagatccgcttcccgcgcacgcttggtcccgaggccaagtccttgctttcagggctgctcaagaaggaccccaagcagaggcttggcgggggctccgaggacgccaaggagatcatgcagcatcgcttctttgccggtatcgtgtggcagcacgtgtacgagaagaagctcagcccacccttcaagccccaggtcacgtcggagactgacaccaggtattttgatgaggagttcacggcccagatgatcaccatcacaccacctgaccaagatgacagcatggagtgtgtggacagcgagcgcaggccccacttcccccagttctcctactcggccagcagcacggcctgaggcggcggtggactgcgctggacgatagcttggagggatggagaggcggcctcgtgccatgatctgtatttaatggtttttatttctcgggtgcatttgagagaagccacgctgtcctctcgagcccagatggaaagacgtttttgtgctgtgggcagcaccctcccccgcagcggggtagggaagaaaactatcctgcgggttttaatttatttcatccagtttgttctccgggtgtggcctcagccctcagaacaatccgattcacgtagggaaatgttaaggacttctacagctatgcgcaatgtggcattggggggccgggcaggtcctgcccatgtgtcccctcactctgtcagccagccgccctgggctgtctgtcaccagctatctgtcatctctctggggccctgggcctcagttcaacctggtggcaccagatgcaacctcactatggtatgctggccagcaccctctcctgggggtggcaggcacacagcagccccccagcactaaggccgtgtctctgaggacgtcatcggaggctgggcccctgggatgggaccagggatgggggatgggccagggtttacccagtgggacagaggagcaaggtttaaatttgttattgtgtattatgttgttcaaatgcattttgggggtttttaatctttgtgacaggaaagccctcccccttccccttctgtgtcacagttcttggtgactgtcccaccggagcctccccctcagatgatctctccacggtagcacttgaccttttcgacgcttaacctttccgctgtcgccccaggccctccctgactccctgtgggggtggccatccctgggcccctccacgcctcctggccagacgctgccgctgccgctgcaccacggcgtttttttacaacattcaactttagtatttttactattataatataatatggaaccttccctccaaattct

SEQ ID NO: 23 - DNA, 1715 bp, Homo sapiens
gaattccagcggcggcgccgttgccgctgccgggaaacacaaggaaagggaaccagcgcagcgtggcgatgggcgggggtagagccccgccggagaggctgggcggctgccggtgacagactgtgccctgtccacggtgcctcctgcatgtcctgctgccctgagctgtcccgagctaggtgacagcgtaccacgctgccaccatgaatgaggtgtctgtcatcaaagaaggctggctccacaagcgtggtgaatacatcaagacctggaggccacggtacttcctgctgaagagcgacggctccttcattgggtacaaggagaggcccgaggcccctgatcagactctaccccccttaaacaacttctccgtagcagaatgccagctgatgaagaccgagaggccgcgacccaacacctttgtcatacgctgcctgcagtggaccacagtcatcgagaggaccttccacgtggattctccagacgagagggaggagtggatgcgggccatccagatggtcgccaacagcctcaagcagcgggccccaggcgaggaccccatggactacaagtgtggctcccccagtgactcctccacgactgaggagatggaagtggcggtcagcaaggcacgggctaaagtgaccatgaatgacttcgactatctcaaactccttggcaagggaacctttggcaaagtcatcctggtgcgggagaaggccactggccgctactacgccatgaagatcctgcgaaaggaagtcatcattgccaaggatgaagtcgctcacacagtcaccgagagccgggtcctccagaacaccaggcacccgttcctcactgcgctgaagtatgccttccagacccacgaccgcctgtgctttgtgatggagtatgccaacgggggtgagctgttcttccacctgtcccgggagcgtgtcttcacagaggagcgggcccggttttatggtgcagagattgtctcggctcttgagtacttgcactcgcgggacgtggtataccgcgacatcaagctggaaaacctcatgctggacaaagatggccacatcaagatcactgactttggcctctgcaaagagggcatcagtgacggggccaccatgaaaaccttctgtgggaccccggagtacctggcgcctgaggtgctggaggacaatgactatggccgggccgtggactggtgggggctgggtgtggtcatgtacgagatgatgtgcggccgcctgcccttctacaaccaggaccacgagcgcctcttcgagctcatcctcatggaagagatccgcttcccgcgcacgctcagccccgaggccaagtccctgcttgctgggctgcttaagaaggaccccaagcagaggcttggtggggggcccagcgatgccaaggaggtcatggagcacaggttcttcctcagcatcaactggcaggacgtggtccagaagaagctcctgccacccttcaaacctcaggtcacgtccgaggtcgacacaaggtacttcgatgatgaatttaccgcccagtccatcacaatcacaccccctgaccgctatgacagcctgggcttactggagctggaccagcggacccacttcccccagttctcctactcggccagcatccgcgagtgagcagtctgcccacgcagaggacgcacgctcgctgccatcaccgctgggtggttttttacccctgcc

SEQ ID NO: 24 - DNA, 1803 bp, Mus musculus
ccttcggaagacttatttttggggctccgcgcccgggcgcccggacgccgcacagccggtgctggagactgaattcggggcttcacaggagtgaataagcactgtgccagtgagctttgttgccagtcctggaacactgttcttcaccggcaggagagtggaaggggttccatgatggagtttgaacattacctcaagaggtttcatattttggatttgtgaattatatttaccctctgctgagaactttgaaacttcagactcaatttctgtccttcaagacattaaatgcagagggattgtatcatggactacaaggagagctgcccaagtgtaagcattcccagctctgacgaacacagagagaaaaagaagaggttcacggtttataaagttctggtctctgtgggcagaagcgagtggtttgtcttcaggagatacgcagagtttgacaaactttacaattctttaaagaagcagtttcctgctatggctctgaagattcctgccaagagaatatttggtgataattttgatccagattttattaaacaaagaagagcaggattgaatgagttcattcagaacttggtcagatatccagagctttacaaccatccagatgtccgagcattccttcaaatggacagcccaagacatcagtcagatccatctgaagatgaggatgaaagaagtacttcgaagccacattctacctcacggaacatcaacctgggaccaactggaaatcctcatgctaaaccaactgacttcgattttttaaaagttattggaaagggcagctttggcaaggttcttcttgcaaaacggaaactggatggaaaattttatgctgtcaaagtgttacagaaaaaaatagttctcaacagaaaagagcaaaaacatattatggctgaacgcaatgtgctcttgaaaaatgtgaagcacccatttttggttggattgcactattctttccaaacaactgaaaagctttattttgttctggattttgttaatggaggggagctcttctttcacctccaaagggaaaggtcttttcctgaacccagagcgaggttttatgccgcggagatcgccagtgccttgggctacctgcactccatcaaaatagtgtacagagacttgaagccagaaaatattcttttggattcaatgggacatgttgtcttaacggattttggactttgcaaagaaggaatcgctatttctgataccaccacaactttttgtggtacaccagagtaccttgcacctgaagtaatcagaaaacagccctatgacaacactgtggactggtggtgcctgggcgctgttctgtatgagatgctgtacgggctgcctcctttttactgccgagatgttgctgaaatgtatgacaatattcttcacaagcccttaaacttgagaccaggagtgagtctcaccgcctggtccattctggaagaacttctagaaaaaaacagacaaaatcgacttggtgccaaagaagactttcttgaaatccagaatcatcctttttttgagtcactcagctggactgacctcgtacaaaaaaagattccacctccatttaaccctaatgtggctggaccagatgatatcagaaactttgatgccgtcttcactgaagaaacggttccctattcagtgtgtgtgtcttctgactattccatcgtgaatgccagtgttctggaggcagatgatgcatttgttggtttttcttacgcccctccttcggaagacttatttttgtgaacactttgacattcagaaaccaat

SEQ ID NO: 25 - DNA, 5227 bp, Mus musculus
ccacgcgtccgcggagagatcgtaccggggttgcggactccggaggtggccacgccgtccagtccagcccccgcccgatcacccgaagaaccaagccggccctgggcagtgacggggttcgagtgaccatggagagcgccttgactgcccgagaccgggtaggggtgcaggactttgtcctgctggagaatttcaccagtgaggctgccttcattgagaacctccggcggcggttccgggagaacctcatttatacctacatcggtcctgtcctagtctctgtcaatccctaccgagacctacagatctacagccggcagcatatggaacgctaccgtggtgtcagtttctatgaagtaccacctcatttgtttgcagtggctgacactgtataccgggcacttcgtactgagcgtcgggaccaggcagtgatgatttctggagagagtggggcaggcaagacagaggccaccaagagactgctccagttctatgcagagacctgcccagcccctgaacggggtggcgcagtgcgagaccgcctgttgcagagcaaccccgtgttagaggcctttgggaatgccaagactctccgcaacgataactccagccggtttggaaagtacatggatgtgcagtttgacttcaagggtgcccccgtgggaggccacattctcagttacctcctggaaaagtcccgggtggtgcaccaaaatcacggagagcggaacttccacgtcttttaccagctactggaggggggcgaggaggagactctccgtcggctgggcttggaacggaacccccagagctacttgtacctggtgaagggccagtgtgccaaggtctcctccatcaacgacaagagtgactggaaggttatgaggaaggcgctgtccgtcattgacttcactgaggatgaagtggaggacttgctcagcatcgtggccagcgtcctacatctgggcaacatccactttgctgctgacgaggacagcaatgcccaggttactactgagaaccagctcaaatatctgaccaggctccttggtgtggaaggtacaacacttagggaagccctgacccacaggaagatcatcgccaagggggaagagctcctgagcccactgaaccttgaacaggcggcatatgcaagggatgcgcttgccaaggctgtgtacagccggacattcacctggctggtcagaaagatcaataggtcactggcctctaaggacgctgagagccccagctggcgaagcaccacggttcttgggctcctggacatttacggctttgaagtgtttcagcataacagcttcgagcagttctgcatcaactactgcaatgagaagctgcagcagctcttcatcgagctgactctcaagtcggagcaggaggaatacgaggctgagggcatcgcgtgggaacctgtccagtacttcaacaacaagatcatctgtgacctggtagaggagaagttcaagggcatcatctccatcttggatgaagagtgcctgcgtcctggggaggccacggacctgacctttctggagaagttggaggacactgtcaagccccaccctcacttcctgacgcacaagctcgctgaccagaagaccaggaaatctctagaccgaggggagttccgccttctgcattatgctggagaggtgacctacagtgtgactgggtttctggataaaaacaatgacctcctcttccggaacctgaaggagaccatgtgcagctcaatgaaccccatcatggcccagtgctttgacaagagtgagctcagtgacaagaagcggccagagacggtggccacccagttcaagatgagcctcctgcagctcgtggagatcctgaggtctaaggagcctgcctatatccggtgcatcaagccaaacgacgccaagcagccgggtcgctttgatgaggtgctcatccgacatcaggtgaagtacctgggactgatggagaatctgcgcgtgcgcagagctggctttgcctatcgtcgcaaatatgaggctttcctgcagaggtacaagtcactgtgcccagagacatggcccatgtgggcaggacggccccaggatggtgtggccgtgttggtcagacacctcggctacaagccagaagagtacaaaatgggcaggactaagatcttcatccgatttcccaagaccttgtttgccacagaggactccctggaagtccggcggcagagtctagccaccaagatccaggcggcctggaggggctttcattggcgacagaaatttctccgggtgaagcgatcagccatctgtatccagtcatggtggcgtggcacactgggccggaggaaggcagccaagaggaagtgggcagcccagaccatccgtcgactcatccgtggcttcattttgcgccattcaccccggtgccctgagaatgccttcttcttggaccacgtgcgcgcctcatttttgcttaacctgaggcggcaactgccccggaatgttctggacacctcctggcccacacccccacctgccctgagagaggcctcagaactgctacgggaactgtgcatgaagaacatggtgtggaagtactgccggagcatcagccctgagtggaagcagcagctgcagcaaaaggcggtggctagtgaaattttcaagggcaagaaggacaactacccccagagtgtccccagactcttcattagcacacggcttggcacagaggagatcagccccagagtgcttcaatccttgggctctgaacccatccagtatgccgtgcccgtggtaaaatacgaccgtaagggttacaagcctcgcccccggcagctgctgctcacgcccagtgctgtggtcattgtggaggatgctaaagtcaagcagagaattgattatgccaacctaaccggaatctctgtcagtagcctgagtgatagcctatttgtgcttcacgtgcagcgtgaagacaacaagcagaagggagatgtggtgctgcagagtgatcatgtgatcgagacactaaccaagacggccctcagtgctgaccgcgtgaacaatatcaacatcaaccagggcagcataacgtttgcagggggtccaggcagggacggcatcattgacttcacatcgggctcagagcttctcatcaccaaggctaagaatggccacctggctgtggtggccccacggctgaattctcggtgatgaaggcttcagtggacccctcctgactcctgatgcttcgcttagtcccctcctcccctcccagttaccaaagactcaagcttccagacagggatccatggacaccctcaaaacccacctgcaaactcctgcctcctgctcgccccctctcgaggtgatcaggagccagggagctaccccatgagtgggccaggccgggccacagcaatagaaaagcagaggcctgagcaggccaggccagccctctgctgatgccaaatatctaagagaagggaattttaactgaggttttctctgagattttttgatgctttataggaaactatttttttaagaaagccattttcctaccctaaacacactggatgtgtttttccctgcctcgaacagggcaaggaatgtaactgaaagactgactgggctgggctggaaggtcctcttctctggccaagcctctcctcattccctgtctgtctgtccatccacctgcaccttttgcagcccactatgacctccaccaaaaggctgaggccacctctgcctaccccatattcctgccttaagaatgtccttttaggggctggggtatagcccagtggtagaactggtgctaagcatgtgtgagaccctgggctcaatccccagcattaaaaaataaaaaataggtttttaatattttcaccccagtctgagggcatccctaaagtgggggaaaagtcttaagagtttggaagtcttcagagacagtgtctggtccaggctcctggaatctacagagctggagacagaggcacacagagggagggaagacttgcctagtagaagactgaagcaaatcctaaagtgaagcccgccctcagcacatctcactgcctttcccagggacagggaggcccataaggcaagggtcgcgtctcatgtatgcacctggctctctgaccagcaatcacccttgggagctaccgggtgggagggactcttctgcctgggtctatgccttaggatgacaacctccatacacatacatactttcgacccaatttaagaatggtagggtcttttattggccttgggtgcctctgtgacctgggagcctagggacagggctggccttggaggaactgcaggggcatcacctctttctgctgcttctctccaccccagaggtccttgggtttgcccagctccctctgtgccctctggggctctcagcccactgctgacacttctgcaatccagagaaacactaaataaagcaatatgtatttgccaacacagtcttcctgtgagtgtggaaaaggggccctagaaggtagacattcttaaggggcttggcactacagaagaaaggagacagacctacttaggagcaatagagagaaaccaagttaggtgtggtattgtgagccttagtgctcaggaagcagggacaggaggattggatttcttagttctaggccagcctggtctacaaatcaagttccagggctatatagacaggcacggggctttggatttgggcaaataaatacctggtctggcagcaccgctggactaaggagacctagcatgggcaatataagcccaggggcctgtgctgatgcaagactcaggtggggagggtcagcacttcataaggaagctggtgtttgaggtatctcaggggcttgcttccagttctggggataaagaatccagtccaaagtggctggagcggtaaaggccacttgtcaacaatggccattttattgtcctggggagatctacttctaggtgatcaaaagacattgttaggaaaatgtcttgggggctagagagatggctcagtggttaagagaactgactgctcttctgaaggtcctgagttcaattcccagcaactacacggtggctcacaaccatctgtaatggggtctgatgccttctgtgtgtctaaagggagcaatggtgatgtactcatatgcataaaataaatgaataaataaacaaatcttaaaaaaaaaaaaaaaaaa

SEQ ID NO: 26 - DNA, 3384 bp, Homo sapiens
tccaagctgaattcgcggccgcgtcgaccacgccggccctgggcagtgacggggttcgggtgaccatggacagtgcgctcaccgcccgtgacagggtgggggtgcaggatttcgtgctgctggagaacttcaccagcgaggccgccttcatcgagaacctacggcggcgatttcgggagaatctcatctacacctacattggccccgtcctggtctctgtcaatccctaccgggacctgcagatctacagccggcaacatatggagcgttaccgtggcgtcagcttctatgaagtgccccctcacctgtttgccgtggcggacactgtgtaccgagcactgcgcacggagcgtcgggaccaggctgtgatgatctctggggagagcggggcaggcaagaccgaagccaccaagaagctgctgcagttctatgcagagacctgcccagccccccaacgcggaggtgccgtgcgggaccggctgctacagagcaacccggtgctggaggcctttggaaatgccaagaccctccggaacgataactccagcaggttcgggaagtacatggatgtgcagtttgacttcaagggtgcccccgtgggtggccacatcctcagttacctcctggaaaagtcacgagtggtgcaccagaatcatggggagcggaacttccacatcttctaccagctgctggaggggggcgaggaagaaactcttcgcaggctgggcttggaacggaacccccagagctacctgtacctggtgaagggccagtgtgccaaagtctcctccatcaacgacaagagtgactggaaggtcgtcaggaaggctctgacagtcattgatttcaccgaggatgaagtggaggacctgctaagcatcgtggccagcgtccttcatttgggcaacatccactttgctgccaacgaggacagcaatgcccaggtcaccaccgagaaccagctcaagtatctgaccaggctcctcagcgtggaaggctcgacgctgcgagaagccctgacacacaggaagatcatcgccaagggggaagagctcctgagcccgctgaacctggaacaggccgcgtacgcacgaaacgccctcgccaaggctgtgtacagccgcacttttacctggctcgtcgggaaaatcaacaggtcgctggcctccaaggacgtggagagccccagctggcggagcaccacggttctcgggctcctggatatttatggcttcgaagtgtttcagcataacagctttgagcagttctgcatcaattactgcaacgaaaagctgcagcagctcttcatcgaactcccgctcaagtcggagcaggaggaatacgaggcagagggcatcgcgtgggaacccgtccagtatttcaacaacaaaatcatctgtgatctggtggaggagaagtttaagggcatcatctcgattttggatgaggagtgtctgcgcccgggggaggccacagacctgaccttcctggagaagctggaggatactgtcaagcaccatccacacttcctgacgcacaagctggctgaccagaggaccaggaaatctctgggccgaggggaattccgccttctgcactatgcgggggaggtgacctacagcgtgaccgggtttctggacaaaaacaatgaccttctcttccggaaccttaaggagaccatgtgtagctcaaagaatcccattatgagccagtgcttcgaccggagcgagctcagtgacaagaagcggccagagacggtcgccacccagttcaagatgagcctcctgcagctggtggagatcctgcagtctaaggagcccgcctacgtccgctgcatcaaacccaatgatgccaaacagcccggccgctttgacgaggtgctgatccgccaccaggtgaagtacctggggctgttggaaaacctgcgtgtgcgcagagctggctttgcctatcgccgcaaatacgaagctttcctgcaaaggtacaagtcactgtgcccagagacgtggcccacgtgggcaggacggccgcaggatggggtggctgtgctggtccgacacctgggctacaagccagaagagtacaagatgggcaggaccaagatcttcatccgcttccccaagaccctgtttgccacagaggatgccctggaggtccggcggcagagcctggccacaaagatccaagctgcctggaggggctttcactggcggcagaaattcctccgggtgaagagatcagccatctgcatccagtcgtggtggcgtggaacactgggccggaggaaggcagccaagaggaagtgggcggcacagaccatccggcggctcatccgaggcttcatcctgcgccacgccccccgctgccccgagaacgccttcttcttggaccatgtgcgcacgtcttttttgctaaacctgaggcggcagctgccccggaatgtcctggacacctactggcccacgcccccacctgccctgcgagaggcctcagagcttctgcgggagttgtgcataaagaacatggtgtggaaatactgccggagtatcagccctgagtggaagcagcagctgcagcagaaggccgtggctagtgagatcttcaagggcaagaaggataattaccctcagagtgtacccaggctcttcatcagcactcggcttggtacagatgagatcagcccccgagtgctgcaggccttgggctctgagcccattcagtatgcggtgcctgttgtgaaatacgaccgcaagggctacaagcctcgctcccggcagctgctgctcacgcccaacgccgtcgtcatcgtggaggacgccaaagtcaagcagaggattgattacgccaacctgaccggaatctctgtcagcagcctgagcgacagtctttttgtgcttcatgtacagcgtgcggacataaagcaaaagggagatgtggtgctgcagagtgaccacgtgattgagacgctgaccaagacagccctcagtgccaaccgcgtgaacagcatcaacatcaaccagggcagcataacgtttgcagggggccccggcagggatggcaccattgacttcacacccggctcggagctgctcatcaccaaggccaagaacgggcacctggctgtggtcgccccacggctgaattatcggtgataaaggcgcccactggaccatcccaacgcccaaagctttgcttttctcctcctccccttcccagttaccaaagagtcgaatttccagacagggacccagggacaccccgaagcccacctgcaatttcccacctcctgcccatccctttcttgagggagcagcaggggccaggagctaccccaggagtgggccaggccgggccacagcaataggaaagccagggccagagcga

SEQ ID NO: 27 - DNA, 19 bp, Mus musculus
acgacgtagccattgtgaa

SEQ ID NO: 28 - DNA, 19 bp, Mus musculus
cgacgtagccattgtgaag

SEQ ID NO: 29 - DNA, 19 bp, Mus musculus
cttcctcctcaagaacgat

SEQ ID NO: 30 - DNA, 19 bp, Mus musculus
ggcaggaagaagagacgat

SEQ ID NO: 31 - DNA, 19 bp, Mus musculus
gacgatggacttccgatca

SEQ ID NO: 32 - DNA, 19 bp, Mus musculus
agcaccgtgtgaccatgaa

SEQ ID NO: 33 - DNA, 19 bp, Mus musculus
ctacttgcactccgagaag

SEQ ID NO: 34 - DNA, 19 bp, Mus musculus
ggatggtgccactatgaag

SEQ ID NO: 35 - DNA, 19 bp, Mus musculus
tggtgccactatgaagaca

SEQ ID NO: 36 - DNA, 19 bp, Mus musculus
ggatgccaaggagatcatg

SEQ ID NO: 37 - DNA, 19 bp, Mus musculus
ccggttctttgccaacatc

SEQ ID NO: 38 - DNA, 19 bp, Mus musculus
ctgacaccaggtatttcga

SEQ ID NO: 39 - DNA, 19 bp, Mus musculus
caccaggtatttcgatgag

SEQ ID NO: 40 - DNA, 19 bp, Mus musculus
ggtatttcgatgaggagtt

SEQ ID NO: 41 - DNA, 19 bp, Mus musculus
tttcgatgaggagttcaca

SEQ ID NO: 42 - DNA, 19 bp, Mus musculus
aacgtggtgaatacatcaa

SEQ ID NO: 43 - DNA, 19 bp, Mus musculus
acgtggtgaatacatcaag

SEQ ID NO: 44 - DNA, 19 bp, Mus musculus
cgtggtgaatacatcaaga

SEQ ID NO: 45 - DNA, 19 bp, Mus musculus
ccatgaatgacttcgatta

SEQ ID NO: 46 - DNA, 19 bp, Mus musculus
ggaggtcatcattgcaaag

SEQ ID NO: 47 - DNA, 19 bp, Mus musculus
gtatttgcactcgagagat

SEQ ID NO: 48 - DNA, 19 bp, Mus musculus
ctcgagagatgtggtgtac

SEQ ID NO: 49 - DNA, 19 bp, Mus musculus
ccgtgacatcaagctggaa

SEQ ID NO: 50 - DNA, 19 bp, Mus musculus
accttatgttggacaaaga

SEQ ID NO: 51 - DNA, 19 bp, Mus musculus
ggtcatggagcatagattc

SEQ ID NO: 52 - DNA, 19 bp, Mus musculus
cacaaggtactttgatgac

SEQ ID NO: 53 - DNA, 19 bp, Homo sapiens
tgagcgacgtggctattgt

SEQ ID NO: 54 - DNA, 19 bp, Homo sapiens
ctgtcatcgaacgcacctt

SEQ ID NO: 55 - DNA, 19 bp, Homo sapiens
tcgaacgcaccttccatgt

SEQ ID NO: 56 - DNA, 19 bp, Homo sapiens
tgaacgagtttgagtacct

SEQ ID NO: 57 - DNA, 19 bp, Homo sapiens
acgagtttgagtacctgaa

SEQ ID NO: 58 - DNA, 19 bp, Homo sapiens
tggcgctgagattgtgtca

SEQ ID NO: 59 - DNA, 19 bp, Homo sapiens
ccagatgcaacctcactat

SEQ ID NO: 60 - DNA, 19 bp, Homo sapiens
gatgcaacctcactatggt

SEQ ID NO: 61 - DNA, 19 bp, Homo sapiens
tgatctctccacggtagca

SEQ ID NO: 62 - DNA, 19 bp, Homo sapiens
caagcgtggtgaatacatc

SEQ ID NO: 63 - DNA, 19 bp, Homo sapiens
agcgtggtgaatacatcaa

SEQ ID NO: 64 - DNA, 19 bp, Homo sapiens
gcgtggtgaatacatcaag

SEQ ID NO: 65 - DNA, 19 bp, Homo sapiens
cagtcatcgagaggacctt

SEQ ID NO: 66 - DNA, 19 bp, Homo sapiens
cttcgatgatgaatttacc

SEQ ID NO: 67 - DNA, 19 bp, Homo sapiens
tggagcacaggttcttcct

SEQ ID NO: 68 - DNA, 19 bp, Mus musculus
ctcaatttctgtccttcaa

SEQ ID NO: 69 - DNA, 19 bp, Mus musculus
aagaagaggttcacggttt

SEQ ID NO: 70 - DNA, 19 bp, Mus musculus
agaagaggttcacggttta

SEQ ID NO: 71 - DNA, 19 bp, Mus musculus
gaagaggttcacggtttat

SEQ ID NO: 72 - DNA, 19 bp, Mus musculus
agaggttcacggtttataa

SEQ ID NO: 73 - DNA, 19 bp, Mus musculus
gaggttcacggtttataaa

SEQ ID NO: 74 - DNA, 19 bp, Mus musculus
ggttcacggtttataaagt

SEQ ID NO: 75 - DNA, 19 bp, Mus musculus
gaagcgagtggtttgtctt

SEQ ID NO: 76 - DNA, 19 bp, Mus musculus
gaacttggtcagatatcca

SEQ ID NO: 77 - DNA, 19 bp, Mus musculus
gatatccagagctttacaa

SEQ ID NO: 78 - DNA, 19 bp, Mus musculus
tccagatgtccgagcattc

SEQ ID NO: 79 - DNA, 19 bp, Mus musculus
gtacttcgaagccacattc

SEQ ID NO: 80 - DNA, 19 bp, Mus musculus
aatcctcatgctaaaccaa

SEQ ID NO: 81 - DNA, 19 bp, Mus musculus
aaccaactgacttcgattt

SEQ ID NO: 82 - DNA, 19 bp, Mus musculus
aacggaaactggatggaaa

SEQ ID NO: 83 - DNA, 19 bp, Mus musculus
tattatggctgaacgcaat

SEQ ID NO: 84 - DNA, 19 bp, Mus musculus
aagaaggaatcgctatttc

SEQ ID NO: 85 - DNA, 19 bp, Mus musculus
tgacaatattcttcacaag

SEQ ID NO: 86 - DNA, 19 bp, Mus musculus
tcgtgaatgccagtgttct

SEQ ID NO: 87 - DNA, 19 bp, Mus musculus
ccgtggtgtcagtttctat

SEQ ID NO: 88 - DNA, 19 bp, Mus musculus
tgaagtaccacctcatttg

SEQ ID NO: 89 - DNA, 19 bp, Mus musculus
agtaccacctcatttgttt

SEQ ID NO: 90 - DNA, 19 bp, Mus musculus
gtaccacctcatttgtttg

SEQ ID NO: 91 - DNA, 19 bp, Mus musculus
agactctccgcaacgataa

SEQ ID NO: 92 - DNA, 19 bp, Mus musculus
gactctccgcaacgataac

SEQ ID NO: 93 - DNA, 19 bp, Mus musculus
ctctccgcaacgataactc

SEQ ID NO: 94 - DNA, 19 bp, Mus musculus
aatcacggagagcggaact

SEQ ID NO: 95 - DNA, 19 bp, Mus musculus
atcacggagagcggaactt

SEQ ID NO: 96 - DNA, 19 bp, Mus musculus
gctacttgtacctggtgaa

SEQ ID NO: 97 - DNA, 19 bp, Mus musculus
acgacaagagtgactggaa

SEQ ID NO: 98 - DNA, 19 bp, Mus musculus
agagtgactggaaggttat

SEQ ID NO: 99 - DNA, 19 bp, Mus musculus
gagtgactggaaggttatg

SEQ ID NO: 100 - DNA, 19 bp, Mus musculus
gtgactggaaggttatgag

SEQ ID NO: 101 - DNA, 19 bp, Mus musculus
gttccgccttctgcattat

SEQ ID NO: 102 - DNA, 19 bp, Mus musculus
caggaggattggatttctt

SEQ ID NO: 103 - DNA, 19 bp, Mus musculus
cttaggagcaatagagaga

SEQ ID NO: 104 - DNA, 19 bp, Mus musculus
ctgctgacacttctgcaat

SEQ ID NO: 105 - DNA, 19 bp, Mus musculus
ggtgacctacagtgtgact

SEQ ID NO: 106 - DNA, 19 bp, Mus musculus
tccgacatcaggtgaagta

SEQ ID NO: 107 - DNA, 19 bp, Mus musculus
ctaagatcttcatccgatt

SEQ ID NO: 108 - DNA, 19 bp, Mus musculus
aggcggtggctagtgaaat

SEQ ID NO: 109 - DNA, 19 bp, Mus musculus
ggcggtggctagtgaaatt

SEQ ID NO: 110 - DNA, 19 bp, Mus musculus
agcagagaattgattatgc

SEQ ID NO: 111 - DNA, 19 bp, Mus musculus
attgattatgccaacctaa

SEQ ID NO: 112 - DNA, 19 bp, Mus musculus
ttgattatgccaacctaac

SEQ ID NO: 113 - DNA, 19 bp, Mus musculus
tgccaacctaaccggaatc

SEQ ID NO: 114 - DNA, 19 bp, Mus musculus
acctaaccggaatctctgt

SEQ ID NO: 115 - DNA, 19 bp, Mus musculus
tcatgtgatcgagacacta

SEQ ID NO: 116 - DNA, 19 bp, Mus musculus
tgtgatcgagacactaacc

SEQ ID NO: 117 - DNA, 19 bp, Mus musculus
tcgagacactaaccaagac

SEQ ID NO: 118 - DNA, 19 bp, Mus musculus
ccgcgtgaacaatatcaac

SEQ ID NO: 119 - DNA, 19 bp, Mus musculus
cggcatcattgacttcaca

SEQ ID NO: 120 - DNA, 19 bp, Mus musculus
gcacatctcactgcctttc

SEQ ID NO: 121 - DNA, 19 bp, Mus musculus
tgccttaggatgacaacct

SEQ ID NO: 122 - DNA, 19 bp, Homo sapiens
gatctacagccggcaacat

SEQ ID NO: 123 - DNA, 19 bp, Homo sapiens
tctacagccggcaacatat

SEQ ID NO: 124 - DNA, 19 bp, Homo sapiens
acgacaagagtgactggaa

SEQ ID NO: 125 - DNA, 19 bp, Homo sapiens
agtcggagcaggaggaata

SEQ ID NO: 126 - DNA, 19 bp, Homo sapiens
attccgccttctgcactat

SEQ ID NO: 127 - DNA, 19 bp, Homo sapiens
ttccgccttctgcactatg

SEQ ID NO: 128 - DNA, 19 bp, Homo sapiens
accttaaggagaccatgtg

SEQ ID NO: 129 - DNA, 19 bp, Homo sapiens
ccttaaggagaccatgtgt

SEQ ID NO: 130 - DNA, 19 bp, Homo sapiens
ccatgtgtagctcaaagaa

SEQ ID NO: 131 - DNA, 19 bp, Homo sapiens
gcgagctcagtgacaagaa

SEQ ID NO: 132 - DNA, 19 bp, Homo sapiens
tcgccgcaaatacgaagct

SEQ ID NO: 133 - DNA, 19 bp, Homo sapiens
atacgaagctttcctgcaa

SEQ ID NO: 134 - DNA, 19 bp, Homo sapiens
tacgaagctttcctgcaaa

SEQ ID NO: 135 - DNA, 19 bp, Homo sapiens
ctcggcttggtacagatga

SEQ ID NO: 136 - DNA, 19 bp, Homo sapiens
ggattgattacgccaacct

SEQ ID NO: 137 - DNA, 19 bp, Homo sapiens
gcgtgcggacataaagcaa

SEQ ID NO: 138 - DNA, 19 bp, Homo sapiens
ttgagacgctgaccaagac

SEQ ID NO: 139 - DNA, 19 bp, Homo sapiens
accgcgtgaacagcatcaa

SEQ ID NO: 140 - DNA, 19 bp, Homo sapiens
gttaccaaagagtcgaatt

SEQ ID NO: 141 - DNA, 19 bp, Homo sapiens
agagtcgaatttccagaca